(19) United States
(12) Patent Application Publication
(10) Pub. No.: US 2024/0092844 A1
(43) Pub. Date: Mar. 21, 2024

(54) SEAMLESS INTEGRATION OF ENGINEERED ZINC FINGERS INTO ENDOGENOUS TRANSCRIPTION FACTORS TO COMMANDEER THEIR NATURAL FUNCTIONS

(71) Applicants: New York University, New York, NY (US); The Governing Council of the University of Toronto, Toronto (CA)

(72) Inventors: Marcus NOYES, Monmouth Junction, NJ (US); Mikko TAIPALE, Toronto (CA); Philip M. KIM, Toronto (CA)

(21) Appl. No.: 18/264,207

(22) PCT Filed: Feb. 4, 2022
(86) PCT No.: PCT/US2022/015346

§ 371 Date: Aug. 3, 2023

Related U.S. Application Data
(60) Provisional application No. 63/145,929, filed on Feb. 4, 2021.

(51) Int. Cl. C07K 14/47

(52) U.S. Cl. CPC C07K 14/4702; A61K 38/00

(57) ABSTRACT

Provided are modified proteins comprising an introduced zinc finger DNA binding domain, and polynucleotides encoding the modified proteins. The modified proteins have a changed DNA binding location relative a DNA binding location of the protein in its unmodified form. The modified proteins include, in addition to the introduced zinc finger DNA binding domain, a gene expression activator domain or a gene expression repressor domain. Methods of using the modified proteins to activate or repress gene expression are also provided. The methods include prophylactic and therapeutic approaches.

### CROSS REFERENCE TO RELATED APPLICATIONS

This application claims priority to U.S. provisional patent application no. 63/145,929, filed Feb. 4, 2021, the entire disclosure of which is hereby incorporated by reference.

### STATEMENT REGARDING FEDERALLY SPONSORED RESEARCH

This invention was made with government support under grant number R01GM118851 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 3, 2022, is named NYU_Zinc_Finger_PCT.txt, and is 77,858 byte in size.

### FIELD

This disclosure generally relates to the field of modulating gene expression responses, and more specifically to modified proteins containing DNA binding domains and transcription activators or transcription repressor domains that function in a natural context.

### BACKGROUND

The precise regulation of gene expression is at the foundation of most biological processes and offers enormous therapeutic potential as the mis-regulation of a gene's expression can be associated with many diseases including cancer, neurodegenerative diseases, and cardiomyopathies. For example, over 660 human genes are estimated to cause diseases due to haploinsufficiency, the effects of which could be corrected by upregulating the functional allele. Conversely, many other diseases are caused by the expression of a gene in the wrong tissue or through gain of function mutations. These diseases could be corrected by downregulating the gene in a tissue specific manner.

Transcription factors (TFs) are endogenous proteins that naturally activate or repress the expression of target genes. These factors modify gene expression by first binding a DNA sequence proximal to the target gene using a DNA-binding domain (DBD) and then recruiting other proteins, through secondary protein interactions, that either modify histones or recruit mediator and/or polymerase components that lead to transcription. These secondary interactions are dictated by other domains within the parent TF which can be common domains such as KRAB domains that repress gene expression, or they can be less common protein sequences the TF has evolved. These effector domains are generically referred to as activation or repression domains. In this way, the DNA-binding specificity of the DBD of the TF determines where the protein will bind in the genome and therefore, which genes will be regulated through the secondary interactions of the effector domains. The most common DBD used by TFs in most metazoans, including human, is the Cys₂His₂ zinc finger (ZF) representing nearly 50% of the human TFs.

Exceptional CRISPR and TALE-based tools have been developed in recent years to modulate gene expression for both academic and therapeutic applications, but some intrinsic characteristics could limit their therapeutic efficacy and their ability to mimic natural regulatory processes. For example, the size of these protein domains limit applications that require AAV delivery. In addition, pre-existing immune responses have been reported in human and primate models for spCas9, while the immune response to the prokaryotic TALE system is unclear but likely. Thus, immunogenicity makes long-term expression of these proteins in humans a significant therapeutic risk. In addition, these prokaryotic proteins require the addition of an activation or repression domain that will function in humans for human therapeutic purposes. This approach requires the expression of a Cas9 or TALE -effector domain fusion that will present the domain out of its natural context. In some cases, the expression of the effector domain out of its natural context can have a significant impact on efficacy. In other cases, the domains employed are not human in origin resulting in a second point of potential immunogenicity. Finally, a TALE-based repressor screen has demonstrated that the position of binding in the genome has a sizeable influence on repression potential, with positions modified by even a few bases having a large impact on efficacy. As a result, applications that require single-base resolution will be limited by the PAM requirement of Cas9. Thus, there is an ongoing and unmet need for improved compositions and methods for precise targeting of DNA locations that modulate a gene's expression while using proteins that minimize the risk of immunogenicity. The present disclosure is pertinent to this need.

### BRIEF DESCRIPTION OF THE FIGURES

FIG. 1. Overview of interface-focused ZF screens. (A) Structure of adjacent ZF domains showing their close proximity. Helical position 6 of domain 1 and position −1 of domain 2 are outlined. (B) Cartoon of interactions between adjacent helices and the DNA. The six helical positions of the three domains are shown as circles with the common contacts made by positions -1, 2, 3, and 6 indicated with arrows. The overlap environment, that includes the base adjacent to the library interaction and the amino acid used to specify that base, is indicated. This environment is unique for each library. (C) Cartoon of the B1H selections. The 3-fingered protein is expressed as a C-terminal fusion to the omega subunit of RNA polymerase. For each library, ZF domain 2 is randomized at six helical positions and screened for amino acid combinations able to specify each of the 64 possible “NNN” targets. This is done in 64 independent screens. Domains 0 and 1 bind to their known, preferred targets, and thereby present an overlap environment that is unique to the library. Only helices able to bind the target in the unique library overlap environment will recruit the polymerase, activate the reporter, and survive on selective media. (D) (left) The helical residues for domains 0, 1, and 2 are shown for each library screened. Domain 2 contains all possible combinations of the six helical residues. Domain 1 is fixed in the selections but varied by library. The 6^{th} residue of domain 1 is the side chain that will be exposed at the interface between domains 1 and 2. Domain 0 is the same in all libraries except library 1. (right) There are 64 DNA targets for domain 2 to be screened against in 64 independent selections. The fixed targets for domain 1 of each library are shown with the overlap base in bold. (E) (left) To assay the success of each selection we determined clusters from the data and used the maximum information content at one position of a cluster to provide a relative measure of enrichment across all selections. (right) Molecular dynamic simulations were performed on all domain 1 helices in their previously characterized contexts. The number of suggested contacts between domain 1 and the DNA are shown for each library. The sequences shown in FIG. 1 are NSTALQARNDSR (SEQ ID NO:1) F1b-domain10-target, NNNACAAAG (SEQ ID NO:2), F1c-zfdomain210 NNNACAAAG (SEQ ID NO:3), F1d-lib1-helix RSDNRA (SEQ ID NO:4), F1d-lib2-helix, QLATSN (SEQ ID NO:5), F1d-lib3-helix, DQSNTR (SEQ ID NO:6), F1d-lib4-helix, FQSGIQ (SEQ ID NO:7), F1d-lib5-helix, HKRNTD (SEQ ID NO:8), F1d-lib6-helix, DQSALG (SEQ ID NO:9), F1d-lib7-helix, TKQNTH (SEQ ID NO:10), F1d-lib8-helix, QLATSY (SEQ ID NO:11), F1d-lib9-helix, RNGNTR (SEQ ID NO:12), F1d-lib10-helix, and YQPNIN (SEQ ID NO:13).

FIG. 2. Specificity solutions are library-specific. (A) (top) A dot plot comparison of 1-Hamming distance is provided comparing the similarity of helical strategies enriched in libraries 1 thru 9 for three G-rich targets (right) and three G-poor targets (left). The darkness of the dot represents the similarity of the enriched populations with dark dots being more similar. Empty spots indicate a failed target selection for one or both of the libraries compared. (bottom) Normalized hamming distance for all libraries across all targets listed from least similar (left) to most similar (right). The targets compared above are underlined in yellow for G-poor targets and blue for G-rich targets. (B) Clusters were determined by MUSI from the enriched helices in each library selection. Three clusters are shown for 4 different binding sites (CCA, TTT, CCG, and GAG). If a cluster was enriched in a library selection the corresponding box is filled black in the table. (C) Schematic illustration (top) and molecular dynamics snapshot (bottom) of the hydrogen bonds between the arginine at position 2 of the domain 2 helix QsR, followed by Ytt with the G* of the CCG* target when an asparagine is at position 6 of the adjacent finger (Library 2 environment) or when an arginine is at position 6 of the adjacent finger (Library 3 context). (D) (top) Cartoon of B1H 2-finger selections. (bottom) The number of helices enriched in the 2-finger selections is shown as a factor of the number of single finger libraries they originated in. (E) A comparison of the helices enriched in the 2-finger selections shows the average number of single finger libraries from which a helix originated in by binding site.

FIG. 3. An interface-focused zinc finger design model. (A) The model is composed of two modules that are trained on single-helix B1H selections to predict residues in partially masked helices that bind 4-mer nucleotide sequences. (B) The generated residues embeddings from these modules are fed into a third module that learns inter-helix compatibility. The full model is trained on two-helix B1H selection data to predict residues in partially masked helix pairs that bind 7-mer nucleotides sequences.

FIG. 4. Performance of two-helix design model. (A) Training and validation accuracy during pre-training step. (B) Training and validation accuracy during fine-tuning step. (C) Helix sequence reconstruction accuracy with different numbers of masked residues. The bars in each group are, from left to right, 6, 8, 10, and 12 residues masked. (D) Comparison of differences between predicted and real selection logos using the developed model and ZFPred. (E) Comparison of differences between predicted and real selection logos using the two-helix model and concatenated logos from the single-helix design model. (F) Comparison of differences between predicted and real selection logos using the two-helix model and concatenated logos from the single-helix B1H selections. (G) Predicted logos, real B1H logos, and concatenated single-helix B1H logos for test set sequences.

FIG. 5. Zinc Finger Designed Nucleases (A) ZFNs bind DNA as dimers in a tail-to-tail orientation, spaced by 5 or 6 bp. The cartoon shows each monomer with two pairs of ZFs separated by a base-skipping linker, for a total 8-finger ZFN. (B) A comparison of loss of fluorescence in a GFP disruption assay for 8-finger ZFNs that were either selected (left bar of each pair) or designed (right bar of each pair) to cut the same targets. (C) Substitution of 2 of the 8-fingers in designed arrays with selected fingers increase activity. (D) Sixteen 12-finger ZFNs, 6 per monomer, are tested for loss of fluorescence. (E) A six-finger array was designed to bind a repeat sequence on chromosome 14, expressed as a GFP fusion, and visualized by live cell imaging. The sequence shown in FIG. (E) is AGTCGCCCAGCTGGGGGCGGG (SEQ ID NO:14).

FIG. 6. Reprogrammed Transcription Factors. (A) The ZFs of KLF6 are seamlessly replaced with designed ZFs. (B) A GFP reporter is activated with 4 ZF designs to bind the TetO sequence. (C) Comparison of RTFs to the rTetR-VP64 activator using the Tet3 ZF array. (D) The ZFs of 4 KRAB TFs are replaced with the Tet3 ZF array and challenged to repress a constitutive GFP reporter. (E) Repression of endogenous targets with Zim3 RTFs measured by RT-qPCR. (F) Left, relative expression of CDKN1C by KLF6 RTFs with 7 ZF arrays designed to bind sequences upstream of the TSS. Right, a comparison of the CDK #200 array with phosphate modifications at CDKN1C and two off-target sequences. (G) Structure, substitution of a phosphate contacting residue (box) can reduce nonspecific affinity. Right, table of misregulated genes by phosphate modification. Below, a comparison of RNA-seq data for 0 and 8 phosphate-contacting modifications.

FIG. 7. Comparison of DNA-binding domain size and relation to DNA. Left, X-ray crystal structure of spCas9 bound to DNA⁽¹⁰⁾. Right, Structure of zinc fingers bound to DNA⁽¹¹⁾. Arrows indicate approximate distance between the C-terminus of the domain and the bound DNA.

FIG. 8. Zinc finger interface and common selection strategies. A. Cartoon of two adjacent fingers interacting with DNA. The six positions of the helix with base-specifying potential are shown. Position 4 is not shown as it is typically a hydrophobic residue that packs into the core of the domain. It is not randomized in any selection schemes. The interface and overlap contacts are indicated with an oval. B. Cartoon of a single finger selection approach where all the randomization is on one of the two fingers⁽¹²⁻¹⁹⁾. These were mostly done with an arginine-guanine contact (highlighted) adjacent to the selected finger^{(12-15, 17-19)} or, in one case, where the library was the N-terminal finger⁽¹⁴⁾. On the randomized helix the letter's in bold (CFWY) were not coded for in the OPEN and other zinc finger libraries^{(14, 15, 18, 19)}. C. Two versions of libraries that selected interface interactions are shown. Top. Many of the contacts were fixed with 5 positions incompletely randomized. The bold amino acids were not available in these libraries^{(20, 21)}. Bottom. Another approach randomized more positions but used a smaller subset of amino acids. Only available amino acids are listed⁽²²⁻²⁴⁾. The sequences shown in B are: ACDEFGHIKLMNPQRSTVWYACDEFGHIKLMNPQRSTVWY (SEQ ID NO:15). The same sequence is shown in the top panel of FIG. 8C. Additional sequences in FIG. 8C, bottom panel, are AEKNQRTV (SEQ ID NO:16), ADHNSTV (SEQ ID NO:17), ADHKNQRS (SEQ ID NO:18), NKRS (SEQ ID NO:19), ADHNQRT (SEQ ID NO:20), AEKNQRTV (SEQ ID NO:21), and DHNSTV (SEQ ID NO:22).

FIG. 9. List of libraries and interfaces tested. All libraries screened as described in this disclosure are listed. The helical residues for the zinc finger adjacent to the library (domain 1 as shown in FIG. 1) are shown for each library. The residue presented at the interface (underlined), the overlap base, and the biophysical category of this side chain is noted. Helical enrichment numbers and selection success is also listed. Library 1a and 1b are the same library using a different base at the overlap position. The same is true for library 3a and 3b. In this disclosure these are referred to as libraries 1(A), 1(C), 3(A), and 3(G) to indicated what overlap base was used in the selections. This is why there are 10 libraries and 12 screens. Cartoons are shown to depict what environment is presented to the selected zinc finger in each library with A overlaps on the left, C overlaps on the right, and G overlaps at the bottom. The sequences shown in the table on the top of FIG. 9 are: S3t-library1a , RSDNLRA (SEQ ID NO:23), S3t-library1b, RSDNLRA (SEQ ID NO:24), S3t-library2, QLATLSN (SEQ ID NO:25), S3t-library3a, DQSNLTR (SEQ ID NO:26), S3t-library3b, DQSNLTR (SEQ ID NO:27), S3t-library4, FQSGLIQ (SEQ ID NO:28), S3t-library5, HKRNLTD (SEQ ID NO:29), S3t-library6, DQSALLG (SEQ ID NO:30), S3t-library7, TKQNLTH (SEQ ID NO:31), S3t-library8, QLATLSY (SEQ ID NO:32), S3t-library9, RNGNLTR (SEQ ID NO:95), S3t-library10, and YQPNLIN (SEQ ID NO:33). The sequences shown are S3a-library1a, ARNDSR (SEQ ID NO:34), S3a-library1b, ARNDSR (SEQ ID NO:35), S3a-library2, NSTALQ (SEQ ID NO:36), S3a-library3a, RTNSQD (SEQ ID NO:37), S3a-library3b, RNTNSQD (SEQ ID NO:38), S3a-library4, QIGSQF (SEQ ID NO:39), S3a-library5, DTNRKH (SEQ ID NO:40), S3a-library6, GLASQD (SEQ ID NO:41), S3a-library7, HTNQKT (SEQ ID NO:42), S3a-library8, YSTALQ (SEQ ID NO:43), S3a-library9, RTNGNR (SEQ ID NO:44), and S3a-library10, NINPQY (SEQ ID NO:45).

FIG. 10. 1-Hamming distance dot plot comparison of libraries by target sequence. Comparison of the similarity of all successful selections for the screens of the primary libraries 1 thru 9 for all 64 triplets. As the plot is 1—Hamming distance, the darker the dot, the more similar the selections. An empty space indicates that the selection for one or both of the libraries failed and therefore no comparison can be made. All plots are on a scale of 0.4 to 1 so that comparisons can be made between plots. GNN (vertical) and NNG (horizontal) targets are boxed to highlight how similar these selections are.

FIG. 11. Global Hamming distance comparisons for libraries that present different overlap bases at the interface. a. Hamming distance comparison across all successful selections for library 1(A)-top, 2-middle, and 4-bottom, with the remaining libraries that were successful across most target selections (two-sided Wilcoxon rank-sum test). Libraries 6 and 10 were omitted because of their poor performance. A-overlap libraries are to the left and C-overlap libraries to the right. Libraries 1(A), 2, and 4 all bind adenine at the overlap and for the most part they are more similar to other A-overlap libraries than they are to C-overlap libraries. b. Libraries 1 and 3 are able to bind A or C and A or G, respectively, at the overlap. A comparison of these libraries using A at the overlap demonstrated that the same library with a different base at the overlap is approximately as similar as the comparison to other A overlap selections (two-sided Wilcoxon rank-sum test). c. A comparison of library 9, that uses an arginine-guanine contact at the interface, is significantly more similar to the only other library screened that also placed an arginine-guanine contact at the overlap library 3(G), than compared to any other library screened (two-sided Wilcoxon rank-sum test).

FIG. 12. Promiscuity of G-rich binding. For the helices enriched in the target selections shown we calculated the number of alternative binding sites these helices were also recovered in. Therefore, the target entropy provides a measure of the general specificity or promiscuity of the helices recovered in these selections. The top 15 binding sites produce helices with the most target entropy and these are exclusive composed of GNN and NNG targets. Conversely, there are no GNN or NNG target in the 13 selections with the lowest target entropy and only 2 of the bottom 24.

FIG. 13. Performance of single-helix design modules. a) Training and validation accuracy during pre-training step. b) Helix sequence reconstruction accuracy with different numbers of masked residues. The bars in each group are, left to right, 3, 4, 5, and 6 residues masked. c) Comparison of differences between predicted and real selection logos using the developed model and ZFPred. d) Predicted logos and real B1H logos for test set sequences.

FIG. 14. Attention values in a layer one and head four of modules one and two compared to distances between nucleotides and residues in Zif268. Attention values and distances for the first a) and second b) helix pairs in Zif268. Attention values are represented by the width of the cyan cylinders in the structural figures, with attention values >=0.2 shown.

FIG. 15. Predicted logos, real B1H logos, and concatenated single-helix B1H logos for all test set sequences.

FIG. 16. Zinc finger arrays that target the TetO sequence for both activation (KLF6) or repression (Zim3). Top box, the TetO sequence is listed in the forward (for) and reverse (rev) direction. Two registers of these sequences were used as the target for zinc finger arrays shown below each and numbered Tet1-Tet4. Lowercase letters indicate the base that is skipped between 2-finger modules. Bottom box, the helices used to specify each of the Tet target sequences are listed as they are expressed in the protein from N-term to C-term. Below, the template sequences for where these helices are expressed in the RTFs KLF6 and Zim3 are shown. The sequences shown in FIG. 16 are: S10-target-1xtetofor, GTCTCTATCACTGATAGGGAGA (SEQ ID NO:46), S10-target-tet1for, GTCTCTATCACTGATAGGGAG (SEQ ID NO:47), S10-target-tet2for, TCTCTATCACTGATAGGGAGA (SEQ ID NO:48), S10-target-1xtetorev, TCTCCCTATCAGTGATAGAGAC (SEQ ID NO:49), S10-target-tet3 rev, TCTCCCTATCAGTGATAGAGA (SEQ ID NO:50), S10-target-tet4rev, CTCCCTATCAGTGATAGAGAC (SEQ ID NO:51), S10-protein-tet1for, QKVHLQSRKWTLSVRKGTLQDQYSSLYKRKGDLNKDPSSLRR (SEQ ID NO:52), S10-protein-tet2for, RKYNLLRRRYSLSAQKAHLLSDPSNLRRQKRLLQNWKVDLRK (SEQ ID NO:53), S 10-protein-tet3 rev, RKFNLLRQSNTLRTLKHHLLNTSSGLCHEKRTLLNWKVDLRK (SEQ ID NO:54), S10-protein-tet4rev, QKTHLLTRRDYLTKRKFTLLRQSNDLRKLKQTLQDRRDRLRR (SEQ ID NO:55), S10-zim3scaffoldtet3rev, MNNSQGRVTFEDVTVNFTQGEWQRLNPEQRNLYRDVMLENYSNLVSVGQGETTKPD VILRLEQGKEPWLEEEEVLGSGRAEKNGDIGGQIWKPKDVKESLAREVPSINKETLTTQ KGVECDGSKKILPLGIDDVSSLQHYVQNNSHDDNGYRKLVGNNPSKFVGQQFACDIC GRKFARKFNLLRHTRIHTGEKPFACDICGRKFAQSNTLRTHTKIHTQRPQIPPKPFACDI CGRKFALKHHLLNHTRIHTGEKPFACDICGRKFATSSGLCHHTKIHTQRPQIPPKPFAC DICGRKFAEKRTLLNHTRIHTGEKPFACDICGRKFAWKVDLRKHTKIHSR (SEQ ID NO:56), S10-klf6scaffoldtet3rev, and MDVLPMCSIFQELQIVHETGYFSALPSLEEYWQQTCLELERYLQSEPCYVSASEIKFDS QEDLWTKIILAREKKEESELKISSSPPEDTLISPSFCYNLETNSLNSDVSSESSDSSEELSP TAKFTSDPIGEVLVSSGKLSSSVTSTPPSSPELSREPSQLWGCVPGELPSPGKVRSGTSG KPGDKGNGDASPDGRRRVFACDICGRKFARKFNLLRHTRIHTGEKPFACDICGRKFAQ SNTLRTHTKIHTQRPQIPPKPFACDICGRKFALKHHLLNHTRIHTGEKPFACDICGRKFA TSSGLCHHTKIHTQRPQIPPKPFACDICGRKFAEKRTLLNHTRIHTGEKPFACDICGRKF AWKVDLRKHTKIHL (SEQ ID NO:57).

FIG. 17. Reprogrammed transcription factor sequences with the Tet3 zinc fingers. a) The sequence for the KRAB containing RTFs for repression, coded with the parent protein, the zinc finger array, helices, and base-skipping linker as shown. b) The sequence for the activating RTFs, coded with the parent protein, the zinc finger array, helices, and base-skipping linker as shown. The sequences shown in a) and b) are:

S11a-znf10scaffoldtet3rev
(SEQ ID NO: 58)
MDAKSLTAWSRTLVTFKDVFVDFTREEWKLLDTAQQIVYRNVMLENYKN
LVSLGYQLTKPDVILRLEKGEEPWLVEREIHQETHPDSETAFEIKSSVS
SRSIFKDKQSCDIKMEGMARNDLWYLSLEEVWKCRDQLDKYQENPERHL
RQVAFTQKKVLTQERVSESGKYGGNCLLPAQLVLREYFHKRDSHTKSLK
HDLVLNGHQDSCASNSNECGQTFCQNIHLIQFARTHTGDKSYKCPDNDN
SLTHGSSLGISKGIHREKPFACDICGRKFARKFNLLRHTRIHTGEKPFA
CDICGRKFAQSNTLRTHTKIHTQRPQIPPKPFACDICGRKFALKHHLLN
HTRIHTGEKPFACDICGRKFATSSGLCHHTKIHTQRPQIPPKPFACDIC
GRKFAEKRTLLNHTRIHTGEKPFACDICGRKFAWKVDLRKHTKIHTGEQ
FLTCNQCGTALVNTSNLIGYQTNHIRENAY;
S11a-znf264scaffoldtet3rev,
(SEQ ID NO: 59)
MAAAVLTDRAQVSVTFDDVAVTFTKEEWGQLDLAQRTLYQEVMLENCGL
LVSLGCPVPKAELICHLEHGQEPWTRKEDLSQDTCPGDKGKPKTTEPTT
CEPALSEGISLQGQVTQGNSVDSQLGQAEDQDGLSEMQEGHFRPGIDPQ
EKSPGKMSPECDGLGTADGVCSRIGQEQVSPGDRVRSHNSCESGKDPMI
QEEENNFACDICGRKFARKFNLLRHTRIHTGEKPFACDICGRKFAQSNT
LRTHTKIHTQRPQIPPKPFACDICGRKFALKHHLLNHTRIHTGEKPFAC
DICGRKFATSSGLCHHTKIHTQRPQIPPKPFACDICGRKFAEKRTLLNH
TRIHTGEKPFACDICGRKFAWKVDLRKHTKIHTGKNPISVTDVGRPFTS
GQTSVTLRELLLGKDFLNVTTEANILPEETSSSASDQPYQRETPQVSSL
S11a-znf324scaffoldtet3rev
(SEQ ID NO: 60)
MAFEDVAVYFSQEEWGLLDTAQRALYRRVMLDNFALVASLGLSTSRPRV
VIQLERGEEPWVPSGTDTTLSRTTYRRRNPGSWSLTEDRDVSGEWPRAF
PDTPPGMTTSVFPVAGACHSVKSLQRQRGASPSRERKPTGVSVIYWERL
LLGSGSGQASVSLRLTSPLRPPEGVRLREKTLTEHALLGRQPRTPERQK
PCAQEVPGRTFGSAQDLEAAGGRGHHRMGAVWQEPHRLLGGQEPSTWDE
LGEALHAGEKSFACDICGRKFARKFNLLRHTRIHTGEKPFACDICGRKF
AQSNTLRTHTKIHTQRPQIPPKPFACDICGRKFALKHHLLNHTRIHTGE
KPFACDICGRKFATSSGLCHHTKIHTQRPQIPPKPFACDICGRKFAEKR
TLLNHTRIHTGEKPFACDICGRKFAWKVDLRKHTKIHTGEKTVRRSRAS
LHPQARSVAGASSEGAPAKETEPTPASGPAAVSQPAEV
S11b-klf7scaffoldtet3rev
(SEQ ID NO: 61)
MDVLASYSIFQELQLVHDTGYFSALPSLEETWQQTCLELERYLQTEPRR
ISETFGEDLDCFLHASPPPCIEESFRRLDPLLLPVEAAICEKSSAVDIL
LSRDKLLSETCLSLQPASSSLDSYTAVNQAQLNAVTSLTPPSSPELSRH
LVKTSQTLSAVDGTVTLKLVAKKAALSSVKVGGVATAAAAVTAAGAVKS
GQSDSDQGGLGAEACPENKKRVFACDICGRKFARKFNLLRHTRIHTGEK
PFACDICGRKFAQSNTLRTHTKIHTQRPQIPPKPFACDICGRKFALKHH
LLNHTRIHTGEKPFACDICGRKFATSSGLCHHTKIHTQRPQIPPKPFAC
DICGRKFAEKRTLLNHTRIHTGEKPFACDICGRKFAWKVDLRKHTKIHI
S11b-foxr2scaffoldtet3rev
(SEQ ID NO: 62)
MDLKLKDCEFWYSLHGQVPGLLDWDMRNELFLPCTTDQCSLAEQILAKY
RVGVMKPPEMPQKRRPSPDGDGPPCEPNLWMWVDPNILCPLGSQEAPKP
SGKEDLTNISPFPQPPQKDEGSNCSEDKVVESLPSSSSEQSPLQKQGIH
SPSDFELTEEEAEEPDDNSLQSPEMKCYQSQKLWQINNQEKSFACDICG
RKFARKFNLLRHTRIHTGEKPFACDICGRKFAQSNTLRTHTKIHTQRPQ
IPPKPFACDICGRKFALKHHLLNHTRIHTGEKPFACDICGRKFATSSGL
CHHTKIHTQRPQIPPKPFACDICGRKFAEKRTLLNHTRIHTGEKPFACD
ICGRKFAWKVDLRKHTKIHIQECMSQPELLTSLFDL
S11b-zxdcscaffoldtet3rev
(SEQ ID NO: 63)
MDLPALLPAPTARGGQHGGGPGPLRRAPAPLGASPARRRLLLVRGPEDG
GPGARPGEASGPSPPPAEDDSDGDSFLVLLEVPHGGAAAEAAGSQEAEP
GSRVNLASRPEQGPSGPAAPPGPGVAPAGAVTISSQDLLVRLDRGVLAL
SAPPGPATAGAAAPRRAPQASGPSTPGFACDICGRKFARKFNLLRHTRI
HTGEKPFACDICGRKFAQSNTLRTHTKIHTQRPQIPPKPFACDICGRKF
ALKHHLLNHTRIHTGEKPFACDICGRKFATSSGLCHHTKIHTQRPQIPP
KPFACDICGRKFAEKRTLLNHTRIHTGEKPFACDICGRKFAWKVDLRKH
TKIHSRRQDLLPQLEAPSSLTPSSELSSPGQSELTNMDLAALFSDTPAN
ASGSAGGSDEALNSGILTIDVTSVSSSLGGNLPANNSSLGPMEPLVLVA
HSDIPPSLDSPLVLGTAATVLQQGSFSVDDVQTVSAGALGCLVALPMKN
LSDDPLALTSNSNLAAHITTPTSSSTPRENASVPELLAPIKVEPDSPSR
PGAVGQQEGSHGLPQSTLPSPAEQHGAQDTELSAGTGNFYLESGGSART
DYRAIQLAKEKKQRGAGSNAGASQSTQRKIKEGKMSPPHFHASQNSWLC
GSLVVPSGGRPGPAPAAGVQCGAQGVQVQLVQDDPSGEGVLPSARGPAT
FLPFLTVDLPVYVLQEVLPSSGGPAGPEATQFPGSTINLQDLQ

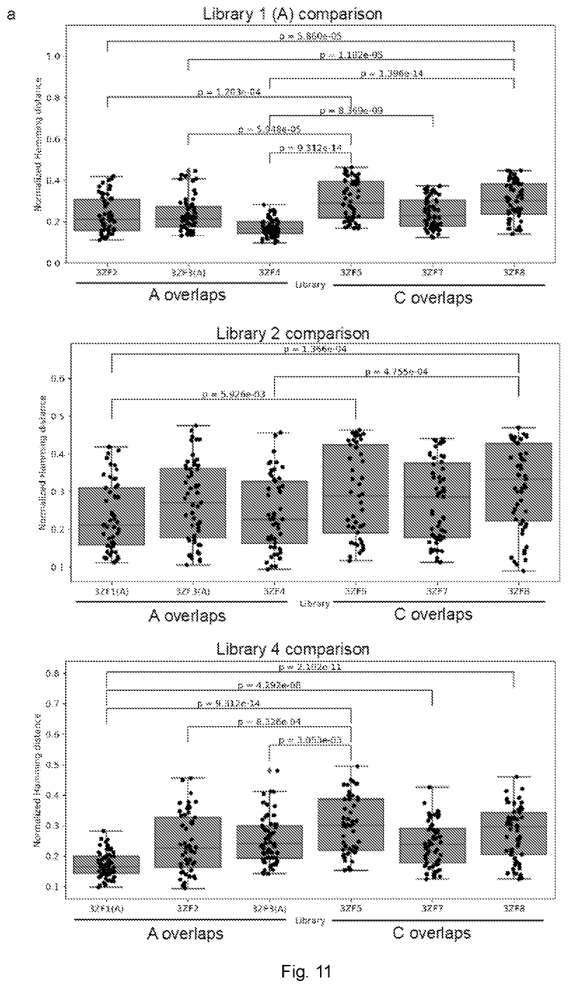

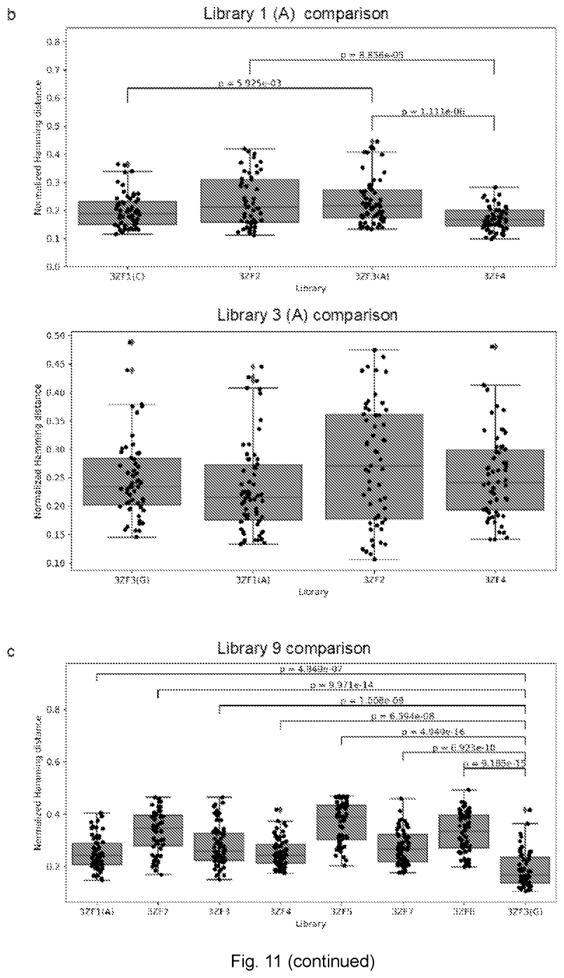

FIG. 18. EGFP repression by ZIM3 RTFs. The zinc fingers of ZIM3 were replaced with the TetO-binding zinc finger arrays described in FIG. 11 and the examples. These were expressed in a HEK293T cell line with EGFP expression driven by a constitutive promoter. EGFP fluorescence relative to controls are shown.

FIG. 19. Repression of endogenous genes with ZIM3 RTFs. a) Four zinc finger arrays were designed to bind sequences near the TSS of DPH1, RAB1a, and UBE4A as shown. The position of the gRNA used by spCas9 is also shown for comparison. b) expression levels as measured by RT-qPCR are shown for each RTF. c) Cartoon and sequence of the DPH1 T2F8 RTF is shown for reference and clarity. In all RTFs, the ZIM3 and ZF scaffold are the same with only the helical residues changing. The sequences shown in FIG. 19 are:

S13c-dph1t2f8zfhelices
(SEQ ID NO: 64)
RKWNLLMRSTNLRDYPYLLRNERSKLRRRVDTLLDHLSNLRKDPSALIR
RLDVLRA,
S13c-dph1t2f8
(SEQ ID NO: 65)
MNNSQGRVTFEDVTVNFTQGEWQRLNPEQRNLYRDVMLENYSNLVSVGQ
GETTKPDVILRLEQGKEPWLEEEEVLGSGRAEKNGDIGGQIWKPKDVKE
SLAREVPSINKETLTTQKGVECDGSKKILPLGIDDVSSLQHYVQNNSHD
DNGYRKLVGNNPSKFVGQQFACDICGRKFARKWNLLMHTRIHTGEKPFA
CDICGRKFARSTNLRDHTKIHTQRPQIPPKPFACDICGRKFAYPYLLRN
HTRIHTGEKPFACDICGRKFAERSKLRRHTKIHTQRPQIPPKPFACDIC
GRKFARVDTLLDHTRIHTGEKPFACDICGRKFAHLSNLRKHTKIHTQRP
QIPPKPFACDICGRKFADPSALIRHTRIHTGEKPFACDICGRKFARLDV
LRAHTKIHSR,
and
S13c-dph1t2f8dnatarget
(SEQ ID NO: 66.)
CTGGTCGTATCCGGGGCAGCGGAGCAGG

FIG. 20. A comparison of the global regulation induced by CDKN1C-targeting zinc finger arrays as RTF and when expressed as fusion to truncated activation domains. a) For the CDK125, 150, 172, and 200 zinc finger arrays we expressed these as KLF6 RTFs (FL) as well as fusions to either the truncated KLF6 transactivation domain (TAD) or VP64. RNA-seq results are shown. b) PCA of RNA-seq results demonstrates that regulated genes mostly cluster by the zinc finger arrays employed, not the mode of activation. c) comparison of common regulated genes shows again that most off-target regulation clusters by which zinc fingers are employed.

FIG. 21. The influence of target C-content and nonspecific affinity. a) The DNA targets for the 4 best arrays designed to activate CDKN1C are shown with CDK200 demonstrating the lowest G-count. b) The helices used by the most promiscuous CDK125 are shown with the position −1 and 6 arginines. These are designed to bind guanine and likely prefer guanine. However, arginines at these helical positions are also able to bind any base at their target positions which likely contributes to the high degree of off-target regulation with arrays designed to bind these G-rich targets. c) RNA-seq results for CDK125 without phosphate modifications. d) RNA-seq results for CDK125 with 8 phosphate contacts modified. e) Table of misregulated genes demonstrates that, despite the G-rich target for CDK125, nearly half of the misregulated genes are lost by reducing the nonspecific affinity. The sequences shown in FIG. 21 are:

(SEQ ID NO: 67)
S15a-target125, GCCAATGGGCGGTGCGCGGGGGCCGGGC,
(SEQ ID NO: 68)
S15a-target150, GGCCGCGGCGGGGCGGGGCAGCGGGGCG,
(SEQ ID NO: 69)
S15a-target172, GGGGCGGCCGCCAATCGCCGTGGTGTTG,
(SEQ ID NO: 70)
S15a-target200, TTGAAACTGAAAATACTACATTATGCTA,
and
S15b-zfa125,
(SEQ ID NO: 71)
KYHLSRDRSTLRRRKDHLRNFPYLLRRLKHHLLRERSKLRRLKQTLQVD
RSTLRR.

FIG. 22. Distribution of target sequences in the training and validation datasets. a) Graph representation of the seven-mer sequences in the training and validation datasets. Nodes represent seven-mers and edges connect nodes representing sequences within two substitutions of each other. Orange nodes are validation set sequences; blue nodes are training set sequences. b) Distances of validation set sequences to training set sequences. c) Distances of test set sequences to training set sequences. d) Distances of all seven-mer sequences to training set sequences. e) Distances of all seven-mer sequences to all sequences against which selections were performed.

FIG. 23. Quantification of the effect of pre-training on model performance. a) Comparison of reconstruction accuracies when the model is pre-trained on single-helix selections and re-trained, re-trained with parameters of the single-helix modules frozen, and not pre-trained. b) Comparison of the perplexities when the model is pre-trained on single-helix selections and re-trained, re-trained with parameters of the single-helix modules frozen, and not pre-trained.

FIG. 24. Impact of number of generated samples on maximum likelihood design using A* or temperature dependent sampling. Error bars show the standard deviation (n=18).

FIG. 25. Sequences showing a KLF6-Zinc finger transcription factor and annotations.

(SEQ ID NO: 72)
MDVLPMCSIFQELQIVHETGYFSALPSLEEYWQQTCLELERYLQSEPCY
VSASEIKFDSQEDLWTKIILAREKKEESELKISSSPPEDTLISPSFCYN
LETNSLNSDVSSESSDSSEELSPTAKFTSDPIGEVLVSSGKLSSSVTST
PPSSPELSREPSQLWGCVPGELPSPGKVRSGTSGKPGDKGNGDASPDGR
RRVHRCHFNGCRKVYTKSSHLKAHQRTHTGEKPYRCSWEGCEWRFARSD
ELTRHFRKHTGAKPFKCSHCDRCFSRSDHLALHMKRHL,
(SEQ ID NO: 73)
HRCHFNGCRKVYTKSSHLKAHQRTH,
(SEQ ID NO: 74)
FKCSHCDRCFSRSDHLALHMKRH,
(SEQ ID NO: 75)
FQCRICMRNFSXXXXLXXHIRTH,
(SEQ ID NO: 76)
FACDICGRKFAXXXXLXXHTKIH,
(SEQ ID NO: 77)
ZXCXXCXXZXXXXXZXXHXXXH,
(SEQ ID NO: 78)
ZXCXXCXXZXXXXXZXXHXXXH,
and
(SEQ ID NO: 79)
MDVLPMCSIFQELQIVHETGYFSALPSLEEYWQQTCLELERYLQSEPCY
VSASEIKFDSQEDLWTKIILAREKKEESELKISSSPPEDTLISPSFCYN
LETNSLNSDVSSESSDSSEELSPTAKFTSDPIGEVLVSSGKLSSSVTST
PPSSPELSREPSQLWGCVPGELPSPGKVRSGTSGKPGDKGNGDASPDGR
RRVFACDICGRKFARKFNLLRHTRIHTGEKPFACDICGRKFAQSNTLRT
HTKIHTQRPQIPPKPFACDICGRKFALKHHLLNHTRIHTGEKPFACDIC
GRKFATSSGLCHHTKIHTQRPQIPPKPFACDICGRKFAEKRTLLNHTRI
HTGEKPFACDICGRKFAWKVDLRKHTKIHL.

FIG. 26. Sequences showing a Zim3-KRAB containing zinc finger transcription factor and annotations. The sequences shown in FIG. 26 are:

(SEQ ID NO: 80)
MNNSQGRVTFEDVTVNFTQGEWQRLNPEQRNLYRDVMLENYSNLVSVGQ
GETTKPDVILRLEQGKEPWLEEEEVLGSGRAEKNGDIGGQIWKPKDVKE
SLAREVPSINKETLTTQKGVECDGSKKILPLGIDDVSSLQHYVQNNSHD
DNGYRKLVGNNPSKFVGQQLKCNACRKLFSSKSRLQSHLRRHACQKPFE
CHSCGRAFGEKWKLDKHQKTHAEERPYKCENCGNAYKQKSNLFQHQKMH
TKEKPYQCKTCGKAFSWKSSCINHEKIHNAKKSYQCNECEKSFRQNSTL
IQHKKVHTGQKPFQCTDCGKAFIYKSDLVKHQRIHTGEKPYKCSICEKA
FSQKSNVIDHEKIHTGKRAYECDLCGNTFIQKKNLIQHKKIHTGEKPYE
CNRCGKAFFQKSNLHSHQKTHSGERTYRCSECGKTFIRKLNLSLHKKTH
TGQKPYGCSECGKAFADRSYLVRHQKRIHSR,
(SEQ ID NO: 81)
LKCNACRKLFSSKSRLQSHLRRH,
(SEQ ID NO: 82)
YGCSECGKAFADRSYLVRHQKRIH,
(SEQ ID NO: 83)
FQCRICMRNFSXXXXLXXHIRTH,
(SEQ ID NO: 84)
FACDICGRKFAXXXXLXXHTKIH,
(SEQ ID NO: 85)
ZXCXXCXXXXXXXXZXXHXXXH,
(SEQ ID NO: 86)
ZXCXXCXXZXXXXXZXXHXXXH,
and
(SEQ ID NO: 87)
MNNSQGRVTFEDVTVNFTQGEWQRLNPEQRNLYRDVMLENYSNLVSVGQ
GETTKPDVILRLEQGKEPWLEEEEVLGSGRAEKNGDIGGQIWKPKDVKE
SLAREVPSINKETLTTQKGVECDGSKKILPLGIDDVSSLQHYVQNNSHD
DNGYRKLVGNNPSKFVGQQLKCNACRKLFSSKSRLQSHLRRHACQKPFE
CHSCGRAFGEKWKLDKHQKTHAEERPYKCENCGNAYKQKSNLFQHQKMH
TKEKPYQCKTCGKAFSWKSSCINHEKIHNAKKSYQCNECEKSFRQNSTL
IQHKKVHTGQKPFQCTDCGKAFIYKSDLVKHQRIHTGEKPYKCSICEKA
FSQKSNVIDHEKIHTGKRAYECDLCGNTFIQKKNLIQHKKIHTGEKPYE
CNRCGKAFFQKSNLHSHQKTHSGERTYRCSECGKTFIRKLNLSLHKKTH
TGQKPYGCSECGKAFADRSYLVRHQKRIHSR.

### BRIEF SUMMARY OF THE DISCLOSURE

The present disclosure relates to the use of activating and repressing transcription factors (TFs), and/or the activation or repression domains from these proteins, e.g., effector domains, many of which use zinc fingers (ZFs) to recognize their DNA targets. Among other aspects, the disclosure provides examples of activators and repressors to seamlessly scaffold designed ZFs in place of the ZFs that occur naturally in these proteins.

In various embodiments, the disclosure accordingly provides modified proteins comprising an introduced ZF DNA binding domain. The introduced ZF DNA binding domain comprises one or more changes to a DNA binding domain that may have been present in the DNA binding domain (or other DBD) of the effector protein domain in an unmodified form, or may be a completely new ZF DNA binding domain. In certain examples, the introduced zinc finger binding domain comprises a substitution of an endogenous ZF domain of the protein. The modified protein thus binds to a different location, e.g., a different DNA sequence, relative to the binding location of the transcription activator or repressor protein in its unmodified form.

The DNA binding domains to which the modified proteins bind can be any DNA binding site that is recognized with specificity by the introduced ZF DNA binding domain. In non-limiting embodiments, the DNA binding location is on a chromosome, organelle DNA, or a plasmid. In embodiments, binding of the modified protein promotes expression of a gene that is operably linked to the DNA binding domain to thereby promote expression of the gene. In an alternative embodiment, binding of the modified protein represses or otherwise inhibits expression of a gene that is operably linked to the DNA binding domain to thereby facilitate inhibition of expression of the gene.

In one representative and non-limiting embodiment, an introduced ZF DNA binding domain is present in a protein that comprises an activator domain that is a Krueppel-like factor 6 (KLF6) protein or functional segment thereof.

In another representative and non-limiting embodiment, an introduced ZF DNA binding domain is present in a protein that comprises a gene expression repressor domain that is a KRAB domain. In one non-limiting example, the KRAB domain is comprised by a Zim3 protein or functional segment thereof.

The disclosure includes modifying the described protein by introducing a plurality of ZF domains. In embodiments, the introduced ZF domains bind with specificity to the same DNA sequence. In alternative embodiments, introduced ZF domains bind to different DNA sequences.

The disclosure includes expression vectors encoding the described modified proteins, as well as cDNAs and RNA, including mRNA, encoding the described modified proteins.

The disclosure also includes pharmaceutical compositions comprising one or more of the modified proteins; one or more mRNAs encoding one or more the modified proteins; and one or more expression vectors encoding one or more of said modified proteins. The disclosure includes administering the described proteins, expression vectors encoding them, and pharmaceutical formulations, to an individual in need thereof. In embodiments, the modified proteins promote expression of a therapeutic gene, and/or a gene that has a prophylactic effect against any disease, condition, or disorder. In alternative embodiments, the modified proteins inhibit expression of a gene, wherein inhibition of the expression of the gene provides a therapeutic or prophylactic effect against any disease, condition, or disorder. In embodiments, administration of the described protein to an individual does not stimulate an immune response, or does not stimulate a deleterious immune response, directed toward the modified protein.

The disclosure also includes a method of making any of the described, modified proteins, by expressing the proteins recombinantly, and optionally isolating the modified proteins from an expression system. The disclosure thus also comprises cells which are programmed to express any one or combination of the described modified proteins.

### DETAILED DESCRIPTION

Unless defined otherwise herein, all technical and scientific terms used herein have the same meaning as commonly understood by one of ordinary skill in the art to which this disclosure pertains.

Unless specified to the contrary, it is intended that every maximum numerical limitation given throughout this description includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

All protein herein include proteins that have from 80.0-99.9% identity across their entire lengths to such proteins. The amino acid or polynucleotide sequence as the case may be associated with each GenBank accession number of this disclosure is incorporated herein by reference as presented in the database on the effective filing date of this application or patent. All combinations or specific proteins, and all combinations of types of proteins, are included in the disclosure. Any protein described herein may comprise or consist of the described protein. In embodiments, a described protein may be linked to or a component of another protein, non-limiting examples of which include nuclease activity, said nucleases including CRISPR-nucleases, recombinases, any nickases, and transposases.

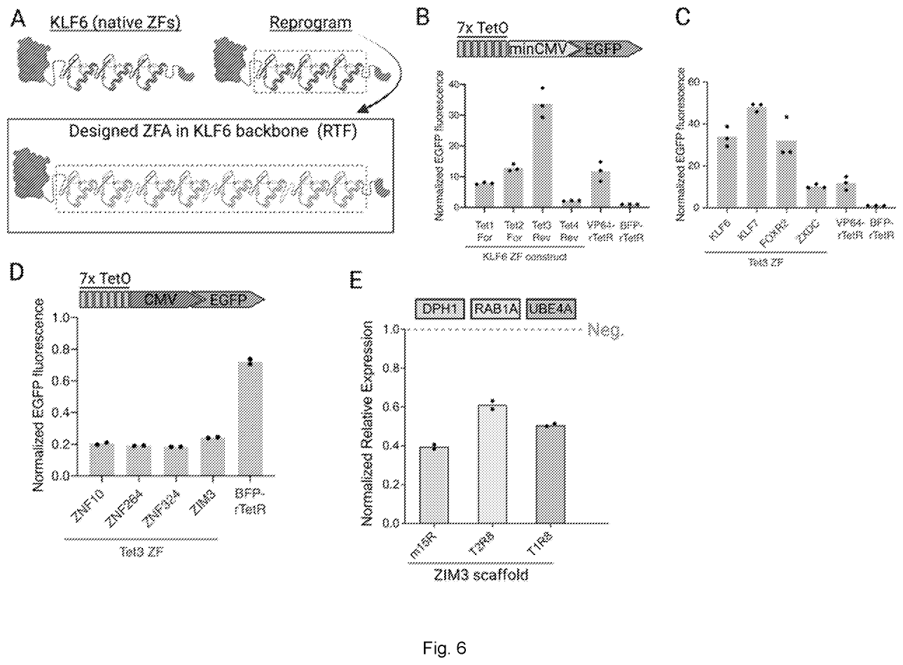

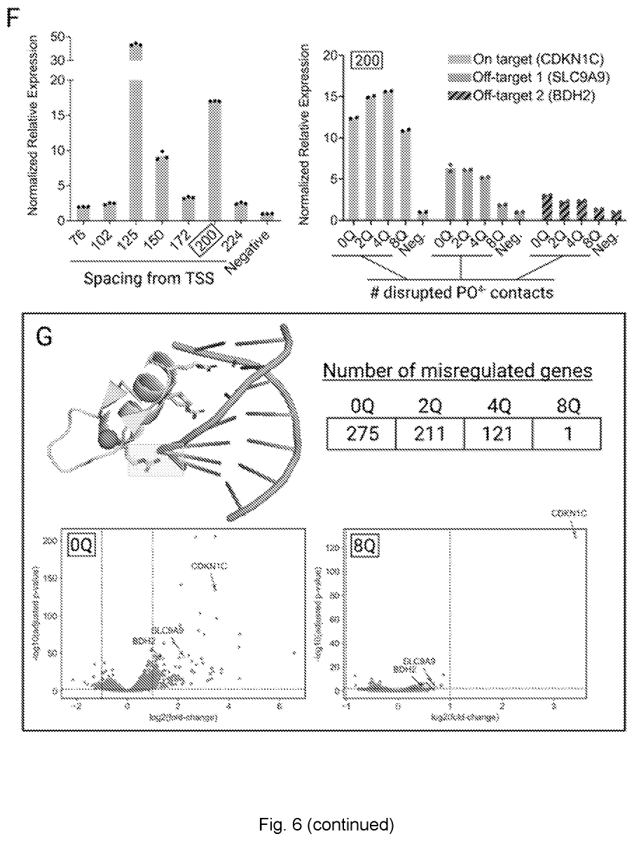

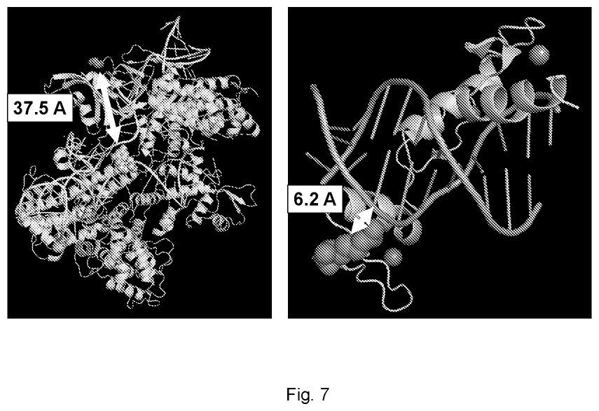

The present disclosure relates to use of effective activating and repressing TFs, and the activation or repression domains from these proteins, including human proteins, many of which use ZFs to recognize their DNA targets. Among other aspects, the disclosure provides examples of activators and repressors to seamlessly scaffold designed ZFs in place of the ZFs that occur naturally in these proteins. By doing so, the disclosure provides for directing the modified protein to any desired DNA sequence in the genome in order to modify proximal gene expression. In this way, the DNA-binding specificity of the TF is effectively reprogrammed to bind alternative sequences in the genome without altering other functions or compositions of the parent protein. Non-limiting embodiments of the disclosure include seamless scaffolds for the KRAB-containing Zim3 protein (repression) and the activating KLF6 protein, and additional examples are described below. These results demonstrate the approach and efficacy of seamless reprogramming that can be applied to any natural ZF or other DBD-expressing TF protein. In an embodiment, the disclosure replaces a DBD that is not a zinc finger with a zinc finger domain. A representative example is provided in FIG. 6c, which demonstrates seamlessly reprograming of FoxR2 which normally uses a DBD referred to as a winged helix. Thus, by using designed ZFs seamlessly scaffolded into proteins that naturally express ZFs or other DBDs, the disclosure includes improving the natural repressing and activating potential of these proteins as the effector domains they harbor will be expressed precisely in their natural context. In addition, in certain embodiments, by using all human components, the disclosure provides for reducing the immunogenic potential of these designer proteins. The proteins generated by seamlessly scaffolding, for example, designed EGR1 zinc fingers into human proteins such as Zim3 (repression) and KLF6 (activation) are used to modify gene expression of disease associated targets. The disclosure includes but is not limited to repression of associated neurodegenerative diseases such as alpha-synuclien and Parkinson's Disease. For activation the disclosure includes but is not limited to SCN5A, a sodium channel where increased expression will overcome multiple disease associated cardiomyopothies. However, the disclosure includes use of this approach to correct the disease associated misregulation of any gene or the correction of pathway function by targeting the activation and/or repression of multiple genes simultaneously. In embodiments, the described proteins inhibit, or promote, expression of a gene, wherein the expression or inhibition of expression provides a prophylactic or therapeutic benefit with respect to any type of cancer.

In embodiments, the disclosure includes the following embodiments, including all embodiments individually and all combinations thereof.

In an embodiment, the disclosure provides a modified protein comprising an introduced ZF DBD, the modified protein having a changed DNA binding specificity relative to a DNA binding specificity of the protein in its unmodified form. In general, the modified protein comprises, in addition to the introduced zinc finger DNA binding domain, a gene expression activator domain or a gene expression repressor domain. An “introduced” zinc finger domain means one or more amino acid changes in an endogenous ZF domain of a protein that changes the DNA binding location of the protein. Thus, an introduced ZF domain comprises a ZF domain that was not present in the protein, prior to modification of the protein as described herein. The introduced ZF domain may include more than one ZF domain. In general, the introduced ZF domain does not change the natural function of the parent protein, e.g., if an activator of transcription includes an introduced ZF domain, the activator of transcription function of the protein is retained, but transcription of a different gene may be promoted. The same rational applies to a repressor. The activators and repressors may bind to any location that is operably linked to any gene. “Operably linked” means binding of the protein is correlated with a change in gene expression, e.g., activation or repression. Thus, the proteins can bind to elements that are proximal to a gene (e.g., a promoter), or elements that are distal from a gene (e.g., an enhancer). Binding to other elements that influence expression of a gene to which the binding site is operably linked are included in the disclosure. In embodiments, the activator or repressor is a transcription factor. Thus, in one embodiment, the activator promotes transcription of mRNA which is in turn translated into a protein. In one embodiment, the repressor inhibits transcription of mRNA. The modified protein of the disclosure may bind to a changed DNA binding location on a chromosome, organelle DNA, or a plasmid. In an embodiment, the DNA binding location is present in the genome of a DNA virus.

In embodiments, any ZF domain that is introduced into a protein as described herein may have the same DBD sequence as any of ZNF324, ZNF264, ZNF10, FoxR2, KLF7, or ZXDC. In embodiments, the ZF domain is a novel sequence.

In embodiments, the gene expression activator domain promotes expression of a gene that is operably linked to the changed DNA binding location relative to the DNA binding location of the unmodified effector protein to thereby provide therapeutic expression of the gene. In a non-limiting embodiment, the gene expression activator domain comprises a Krueppel-like factor 6 (KLF6) protein or functional segment thereof. A “functional segment” means a segment of the protein that is sufficient to promote its activation or repression. In an embodiment, the gene expression repressor domain inhibits expression of a gene that is operably linked to the changed DNA binding location to thereby provide therapeutic inhibition of expression of the gene. In a non-limiting embodiment, the gene expression domain comprises a KRAB domain, wherein the KRAB domain is optionally comprised by a Zim3 protein or functional segment thereof. In a non-limiting embodiment, a modified protein of the disclosure comprises a substitution of an endogenous zinc finger domain of the protein. In embodiments, the introduced zinc finger domain is one of a plurality of zinc finger domains that are introduced into the modified protein to thereby provide a modified protein comprising a plurality of introduced zinc finger domains, and wherein the plurality of introduced zinc finger domains optionally comprise the same changed DNA binding domain. The disclosure also includes cDNAs and mRNAs encoding any modified protein described herein.

In embodiments, the modified protein is encoded by an expression vector, such as an expression vector used to make the modified protein, and/or an expression vector that can be used to deliver the coding sequence to cells so that the cells express the modified protein, which may be for a therapeutic purpose. In non-limiting embodiments, the expression vector may comprise a suitable viral vector, non-limiting embodiments of which include modified viral polynucleotide from an adenovirus, a herpesvirus, or a retrovirus, such as a lentiviral vector. Polynucleotides can be used directly, or they may be introduced into cells using any of a variety of polynucleotide insertion reagents, such as transfection agents. In non-limiting embodiments, a recombinant adeno-associated virus (rAAV) vector may be used. In certain embodiments, the expression vector is a self-complementary adeno-associated virus (scAAV). In embodiments, a composition of this disclosure comprises mRNA encoding one or more of the described modified proteins.

In embodiments, a therapeutically effective amount of a described protein is administered to an individual in need thereof. Administration of the protein includes administration by way of polynucleotides that encode the protein. The term “therapeutically effective amount” as used herein refers to an amount of a described protein to achieve, in a single or multiple doses, the intended purpose of treatment. The amount desired or required will vary depending on the particular protein, its mode of administration, patient specifics and the like. Appropriate effective amounts can be determined by one of ordinary skill in the art informed by the instant disclosure using routine experimentation.

The disclosure also provides a pharmaceutical composition comprising one or more of the described modified proteins, one or more mRNAs encoding one or more of said modified proteins, or one or more expression vectors encoding one or more of said modified proteins. Pharmaceutical compositions generally comprise one or more pharmaceutically acceptable buffers, excipients, and the like.

The disclosure also provides administering one or more described protein to an individual in need thereof. The protein, or a pharmaceutical protein comprising the modified protein, can be administered to the individual using any suitable delivery method.

In embodiments, an individual in need of a described protein is in need of activation or repression of one or more genes. In embodiments, the one or more genes are due to or correlated with a haploinsufficiency.

In embodiments, the described modified proteins do not stimulate an adverse immune response in an individual to which they are introduced. An adverse immune response includes but is not limited to innate immune responses, humoral immune responses, and cell-mediated immune responses, wherein said immune responses are deleterious to the individual. In one embodiment, a described protein does not elicit an increased antibody response that comprises an increase of antibodies that bind to a describes protein, relative to pre-existing antibodies that may bind to the effector domain of a described protein.

While the disclosure relates in part to therapeutic approaches in humans, the described modified proteins can also be used for veterinary purposes, e.g., for non-human animals. Further, the described proteins may be suitable for use in other eukaryotic organisms, such as plants and fungi. In embodiments, the described proteins can be used for prokaryotic purposes.

The disclosure also provides a method of making a described modified protein by modifying a protein to comprise an introduced zinc finger DNA binding domain. In embodiments, the modified protein is produced by cells comprising an expression vector encoding the modified protein, from which the modified protein is separated.

In embodiments, the disclosure includes the described library generation, and analysis of the DNA binding properties of members of the library. In embodiments, one or more methods described herein can be performed by a digital processor and/or a computer running software to perform an algorithm and/or to interpret a signal. In embodiments, the processor runs software or implements an algorithm to interpret an a detectable signal, and may generate a machine and/or user readable output. In embodiments, the digital processor and/or the computer participates in the ZFDesign aspect of this disclosure, as further described below. In embodiments, information obtained by a device or system used to analyze protein binding as described herein can be monitored in real-time by a computer, and/or by a human operator. In embodiments, the processor runs software or implements an algorithm to interpret an optically detectable signal, such as a signal from a detectably labeled protein. In certain embodiments, the disclosure provides as an embodiment or component of the system a non-transitory computer readable storage media for use in performing an algorithm to interpret and/or record signaling events. In embodiments, a system described herein may operate in a networked environment using logical connections to one or more remote computers. In embodiments, a result obtained using a device/system/method of this disclosure is fixed in a tangible medium of expression. The result may be communicated to, for example, a user who produces and/or test modified proteins as described herein.

The following Examples are intended to illustrate but not limit the disclosure.

### EXAMPLES

### Selecting Zinc Finger Specificity and Compatibility

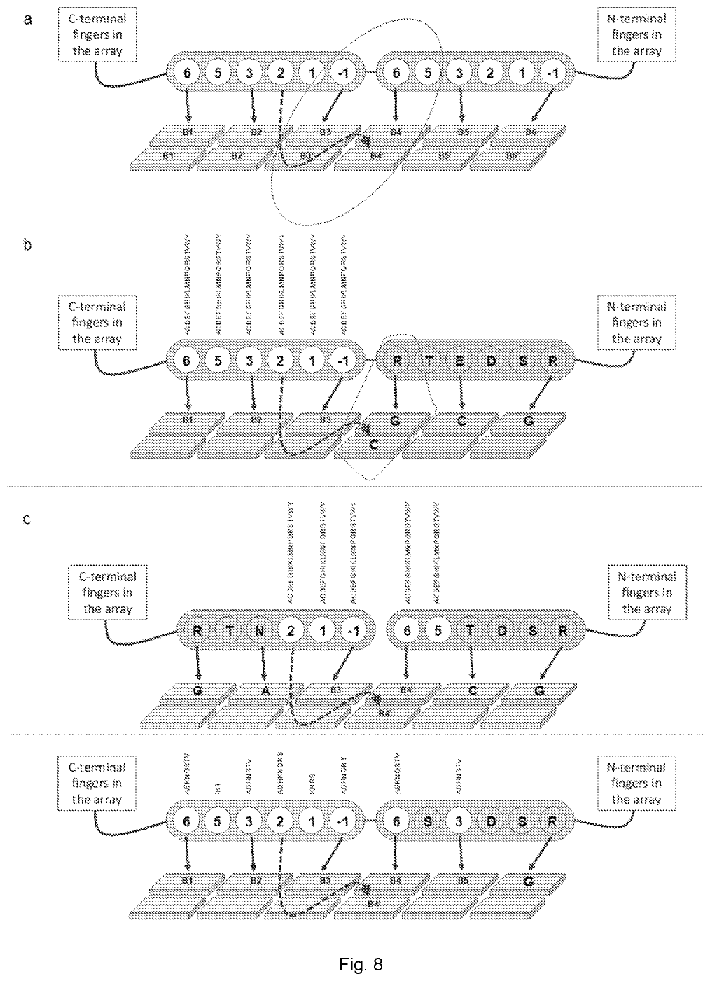

Two general approaches have been used to engineer ZFs with novel specificity (FIG. 8). The first focused on engineering one finger at a time by selecting functional variants from ZF libraries where the 6 base-specifying positions of the helix have been randomized (FIG. 8B). The second approach focused on the interface between adjacent ZFs of an array as the influence that adjacent fingers have on one another has been apparent since the first structures of ZFs bound to DNA were solved (FIG. 8C); this influence of course leads to combinatorially greater complexity, which is a reason for the failure of previous attempts to build a code. While the first approach allows for a comprehensive screen of all amino acid combinations at the six critical positions of the ZF alpha helix^{(24, 26, 27, 29-32)} it only samples these combinations in a single adjacent-finger context. As a result, only ZF strategies enabled by this initial single selection environment are available in subsequent rounds of selection or as the foundation of a ZF model. By contrast, the second approach captures the complexity of compatibility at the interface between ZFs^{(25, 28, 33)} (FIG. 8C). However, as combinatorial explosion quickly exceeds the maximum practical library size for any screening platform, incomplete randomization schemes and the sampling of a limited number of helical positions become necessary. The present disclosure reveals that the solution lies in a combined approach that uses multiple comprehensive libraries in a comprehensive set of interface environments. Thus, in embodiments, each library fully randomizes a single ZF helix in a unique interface environment. Multiple libraries and a diverse, comprehensive set of interface environments would produce broad portfolios of general and interface-specific ZF solutions. The disclosure therefore takes advantage of this interface-derived complexity to provide both the diversity necessary to generate compatible ZF pairs able to bind a wide range of DNA targets, as well as the depth of data required to support a model for ZF array design.

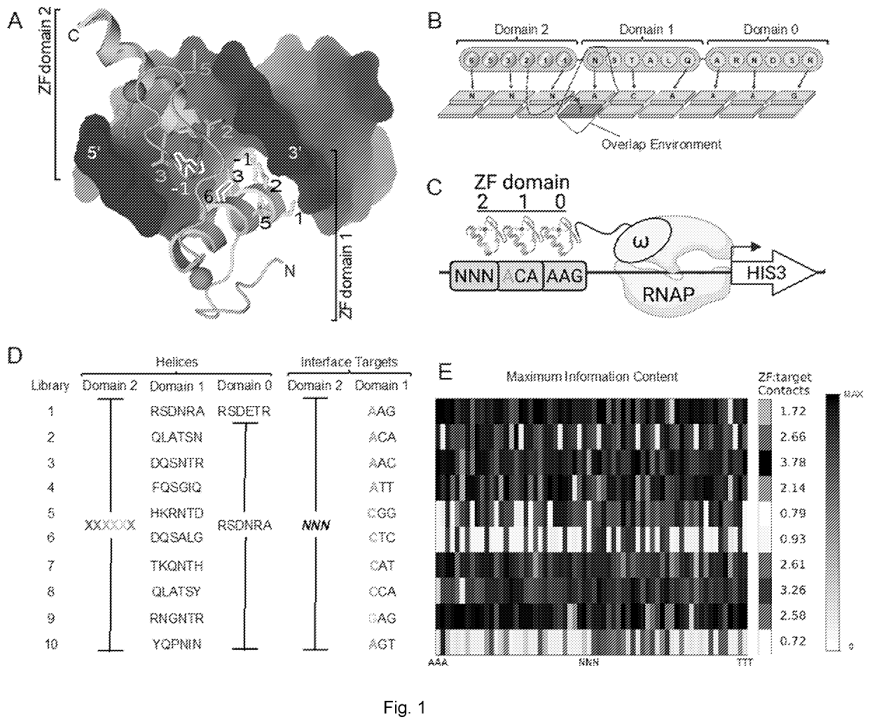

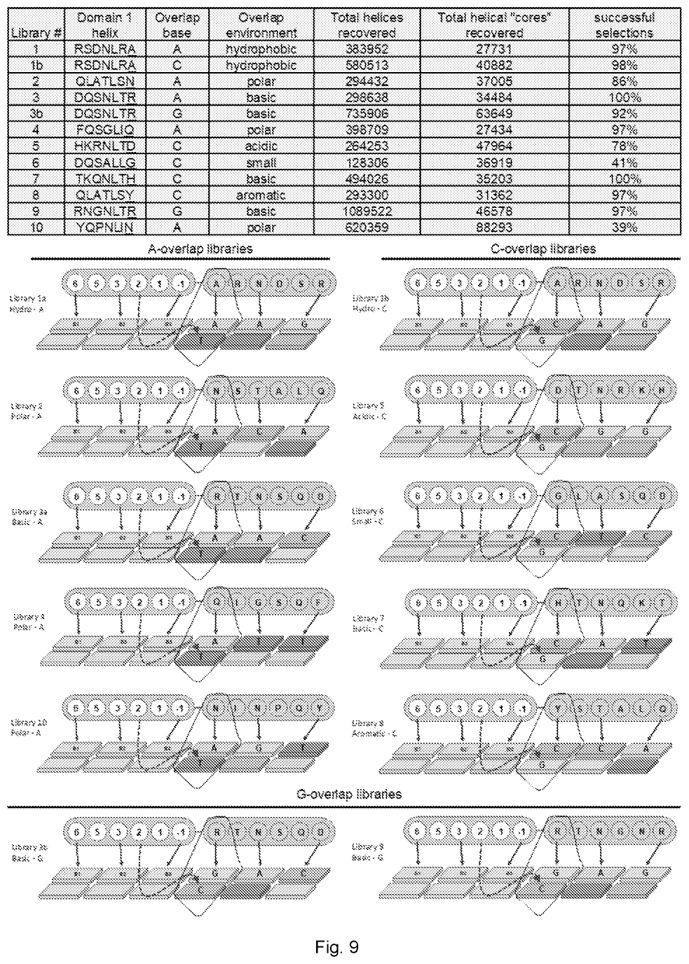

Multiple side chains from adjacent ZFs bind DNA in close proximity to one another; this is especially true at the binding site “overlap” where position 6 of an N-terminal helix can be within hydrogen bonding distance of the position −1 and 2 side chains of its C-terminal neighbor. At this position the specificity of adjacent ZFs overlaps and in this way the N-terminal helix is presenting a specific interface environment to its C-terminal neighbor that is based on the side chain employed and the base specified (FIG. 1A and 1B). Therefore, we screened 10 ZF libraries that each fully randomized the six base-specifying positions of a C-terminal ZF helix using a bacterial hybrid assay (FIG. 1C). Each library puts the random C-terminal ZF helix in a different environment defined by the adjacent ZF helices. We screened these libraries across each of the 64 possible 3 base pair (bp) targets in independent selections to recover functional ZF helices. As the overlap environment should have the greatest adjacent finger influence on the ZF strategies selected in the screens, each library presents a unique interaction between the side chain at position 6 of the adjacent finger and the base it specifies at the overlap (FIG. 1D and FIG. 9). We designed the majority of the libraries to contact adenine or cytosine at the overlap in order to provide a contrast to the arginine-guanine contacts that have been presented at the overlap in the majority of prior ZF screens. In addition, two of the libraries can specify two different bases at the overlap (#1-A, C and #3-A, G). Therefore, we completed two comprehensive screens of these libraries, one screen with each base presented at the overlap. In total, we screened over 49 billion protein-DNA interactions from 10 libraries, across 12 sets of 64 selections per library, for 768 independent selections.

From these screens we found global and target-specific differences between these library contexts, indicative of the strength of the constraint that each context puts upon the C-terminal ZF. The total number of selected helices ranged from 128,000 to over 1 million helices per library screened (FIG. 9). We used MUSI⁽³⁴⁾, a method designed to identify multiple specificities in such data, to define ZF clusters for each library selection and to identify selections with low information content due to failed enrichment. We used the presence of at least one cluster that demonstrates low entropy as our definition of selection success (FIG. 9). To provide a quantitative comparison across all selections we used the maximum information content at a single helical position in any recovered cluster, reasoning that a successful selection should produce clusters where at least one position has been strongly selected for (FIG. 1E). From this analysis we find that libraries were able to enrich helices in 39% to 100% of the 3 bp target selections (FIG. 9). ZF strategies were enriched in over 85% of the 3 bp target selections for 9 of the library screens. In addition, ZF strategies were enriched in at least 8 different library screens for each of the 64 3 bp targets, demonstrating the ability of ZFs to bind any target in a wide range of adjacent finger environments. At least one library that bound either A, C, or G at the overlap successfully enriched helices in over 95% of the selections (libraries 1-A overlap, 7-C overlap, and 9-G overlap), suggesting that ZF strategies exist in a wide variety of contexts independent of overlap base, further underlining the flexibility of the ZF scaffold. We found libraries 6 (C overlap) and 10 (A overlap) to be the least successful libraries (FIG. 9); molecular dynamic simulations suggest that the number of contacts between the adjacent finger (domain 1 in FIG. 1D) employed in each library and the DNA it specifies correlates with global library success, indicating that higher affinity of the neighboring finger enables more ZF strategies (FIG. 1E). Hence, ZF function is significantly impacted by the adjacent finger interaction, while viable ZF binding strategies exist for each overlap base.

### G-rich Binding Modularity and Promiscuity

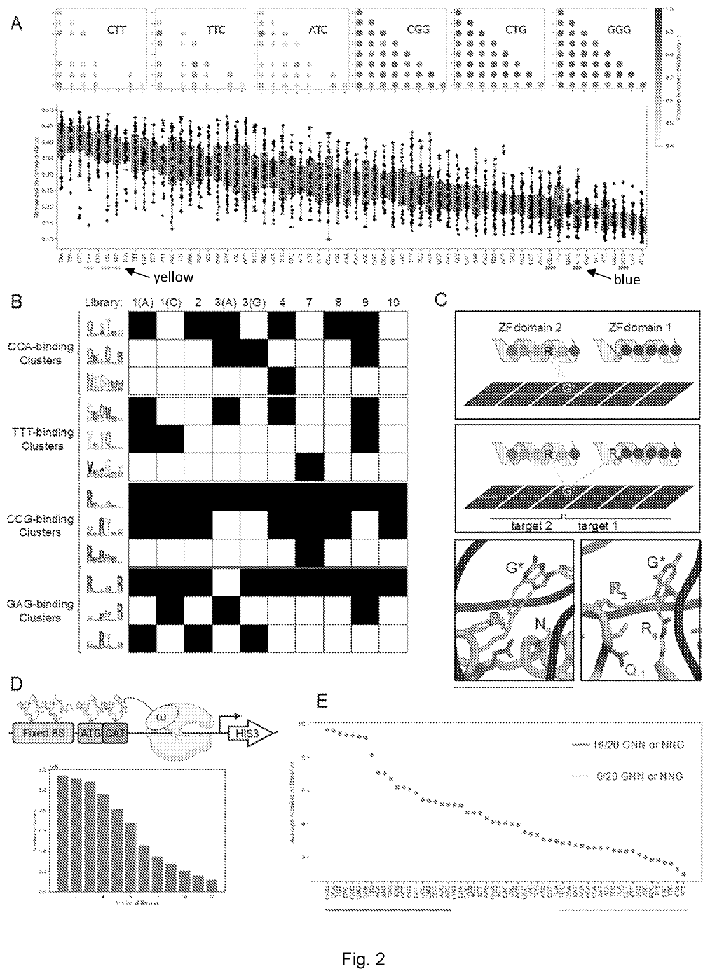

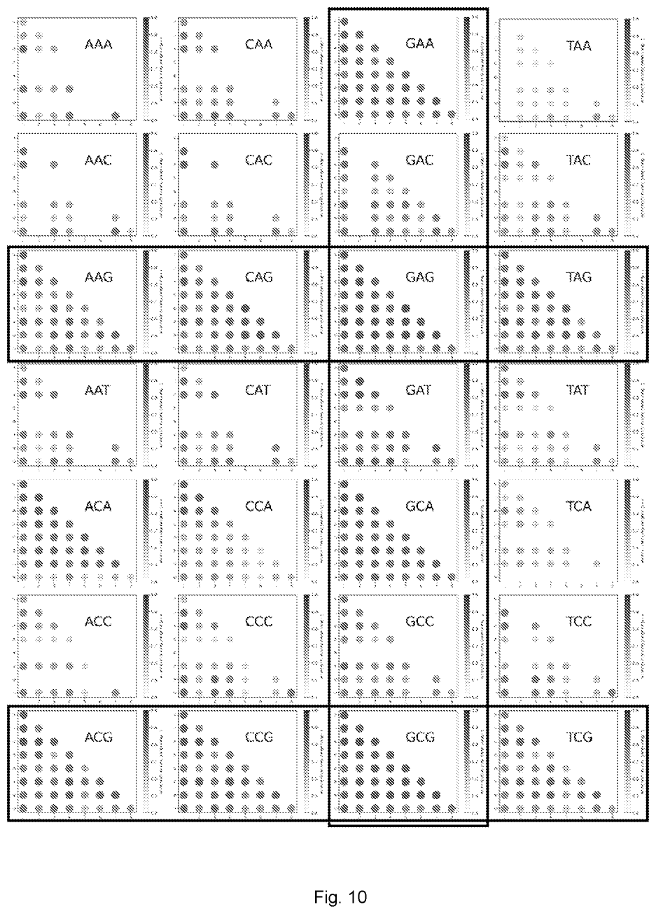

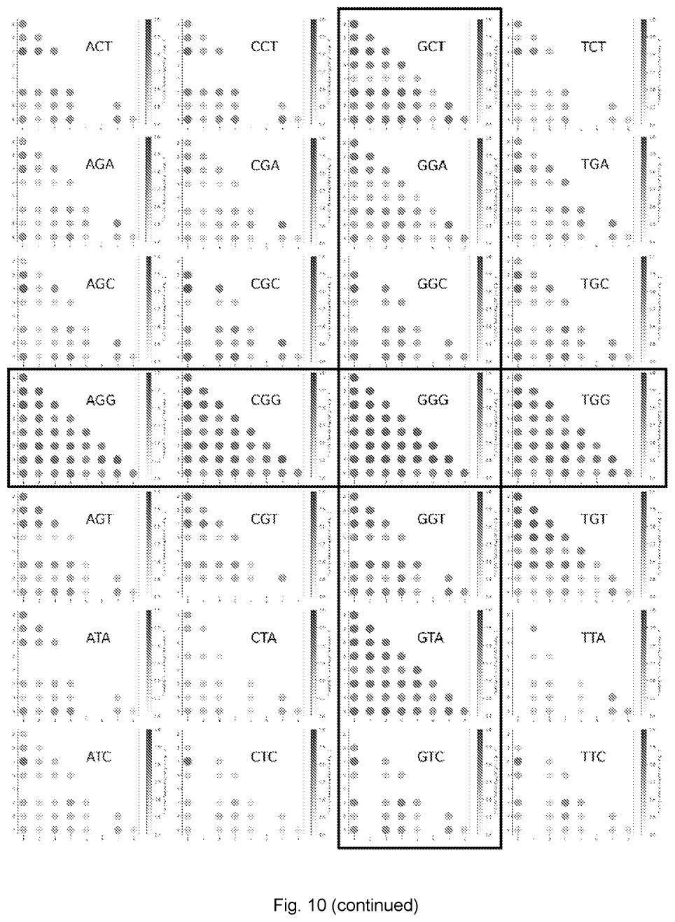

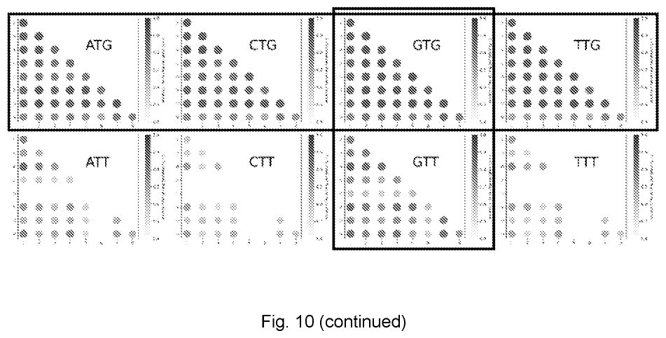

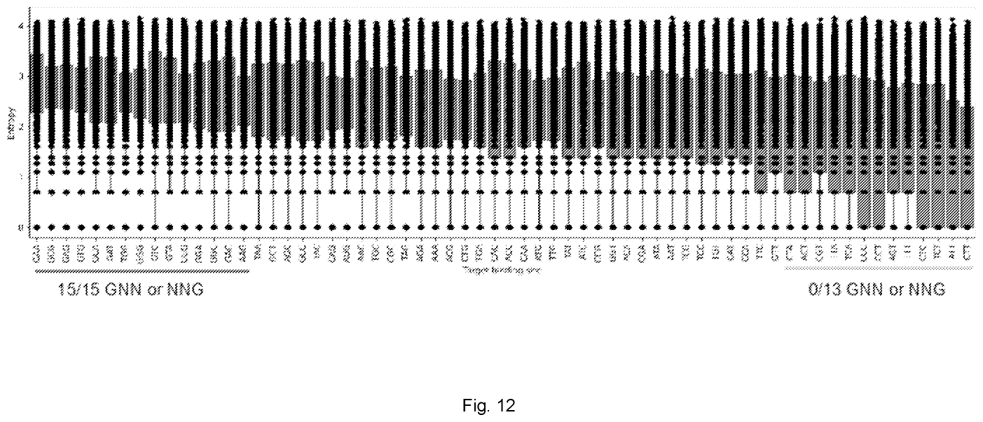

Since the majority of prior ZF selections have been carried out with an arginine-guanine contact presented at the overlap, the disclosure includes libraries that present adenine and cytosine contacts to enrich novel helical strategies. To measure these differences on a global scale we first calculated mean hamming distance between the helices enriched to bind each target across all libraries (FIG. 10). Next, we compared the normalized hamming distance for all targets to compare library differences. While there are general trends that libraries that employ the same overlap base are more similar (FIG. 11), the most striking difference is found when comparing libraries with adenine and cytosine at the overlap to the two libraries that displayed an arginine-guanine contact at the overlap (FIG. 11C). The arginine-guanine contact libraries are more similar to each other than any of the other libraries screened. A comparison of target selection hamming distances across all libraries shows G-rich binding is less influenced by the library context. This suggests that G-rich binding is more modular as these helices appear less dependent on the adjacent finger interaction (FIG. 2A). However, this independence in binding could lead to more promiscuity. To address this possibility we considered helices recovered in each 3 bp target selection and calculated how frequently these helices are recovered in other target selections. The 15 targets with the greatest target selection entropy (i.e., are recovered in the most other selections) all have a G at the GNN or NNG positions where arginine's are the dominant amino acid enriched at the corresponding positions 6 and −1, respectively (FIG. 12). Conversely, none of the 13 targets with the lowest target selection entropy have a G at these positions. These results demonstrate that helices that bind a G at either the first or third position of a binding site are more likely to be promiscuous ZFs. This could help explain why prior selections have largely led to a G-rich bias in ZFs that have been successfully engineered or assembled as modules, with these modules also likely tending towards more off-target binding.

### General and Specialized Binding Strategies

Global differences between library environments were assayed by the success of selections across targets as well as the mean hamming distances. To investigate more specific differences, such as the types of binding strategies enabled by one library environment versus another, we compared the clusters generated by MUSI for each target site selection. For most targets we find general strategies that are common to several successful library selections. We also find specialized strategies that are recovered in a small number of selections and in some cases, only recovered with a single library environment (FIG. 2B). Recovery of helical strategies in one library versus another has been shown to be predictive of activity only in the recovered contexts, confirming that these differences are not due to sampling influences⁽³⁵⁾. In addition, as these differences suggest structural influences at the overlap, we considered whether the presence of a cluster in various library environments might suggest physical influences. Interestingly, in most NCG selections we find a cluster of “QxRYxx” helices (see CCG in FIG. 2B). However, this cluster is not recovered in libraries that presented an arginine from the adjacent finger at the overlap. Molecular dynamics simulations suggest that this is due to a potential competition between the arginine at position 6 of the adjacent finger and position 2 of the selected finger (FIG. 2C).

Data presented in this disclosure demonstrate global and specific differences in ZF function influenced by the adjacent finger environment. While it is believed this data represents the largest screen of ZF function to date, it is still a relatively small number of the potential overlap influences. To test how greater variability at the interface might influence compatibility we created 200 two-finger libraries by assembling pools of helices selected to bind each 3 bp half-site of a 6 bp target. We selected compatible pairs of ZFs from these libraries and analyzed how many starting library environments the helices were enriched from. Most helices enriched in these compatibility assays were only recovered in a minority of the library environments (FIG. 2D). This suggests that despite the fact that all of these helices were pre-selected to bind each half site, only a fraction are enriched in these new environments. When we plot the compatible helices by target selection and assay the number of primary libraries they were recovered in, we again find that G-binding ZFs recovered in the 2-finger selections originate in a large number of the primary libraries while compatible ZFs recovered to bind G-poor targets originate in a small number of selections (FIG. 2E). Together, these results demonstrate that, even for a more comprehensive set of presented environments, the interface has a large influence on ZF function and that G-rich binding helices tend to be more modular and promiscuous. The data from these two-finger library selections provides new insight into the pairwise compatibility of individually functional ZFs.

### A Hierarchical Attention-Based Neural Network Integrates Interface-Derived Selection Data

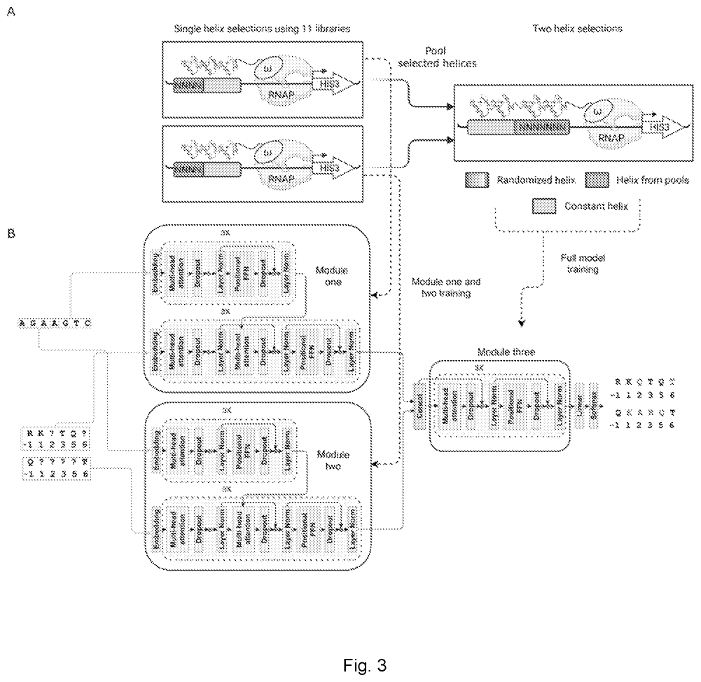

Despite considerable effort, it is considered that all previous attempts at generating a general ZF design code have failed. Given the unprecedented depth of the described screening data, the disclosure includes a novel and unique model that explicitly addresses these neighbor influences. In particular, we separately make use of the single-finger library selections that comprehensively describe single-finger specificity in a variety of neighbor finger contexts and the pair selections that show which ZFs are compatible with each other as neighbors. This information is hierarchical and to make use of it, we developed a novel neural network architecture that implements attention modules in a hierarchical manner (FIG. 3A).

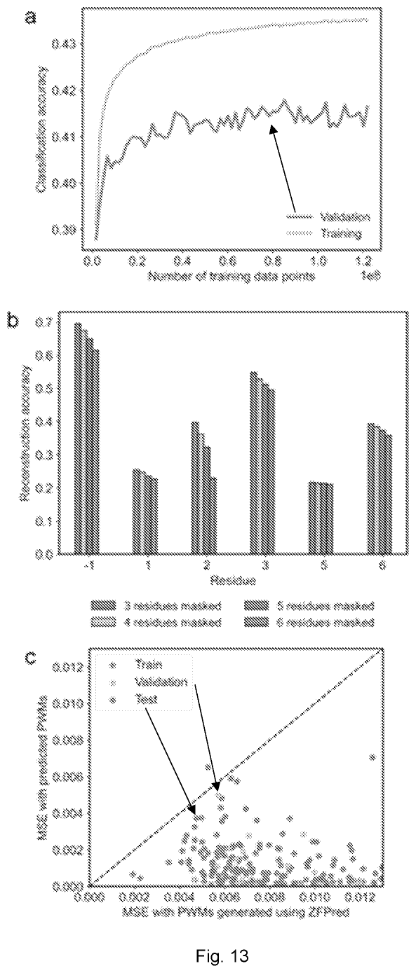

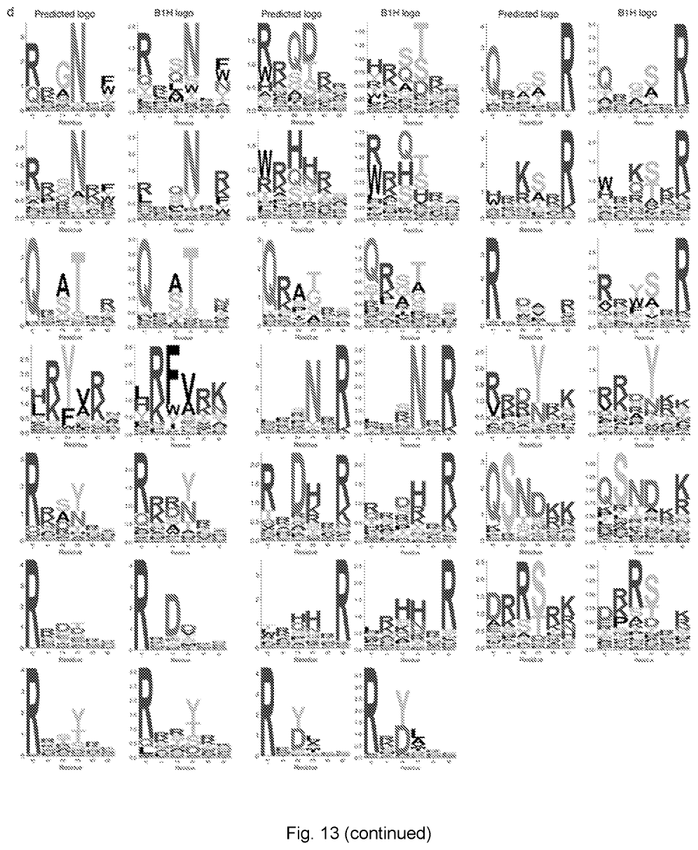

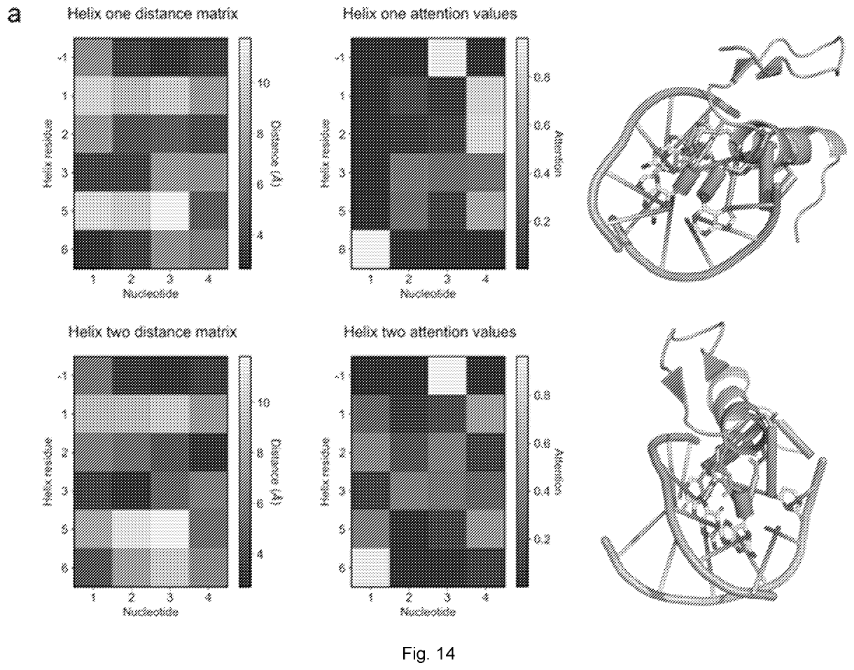

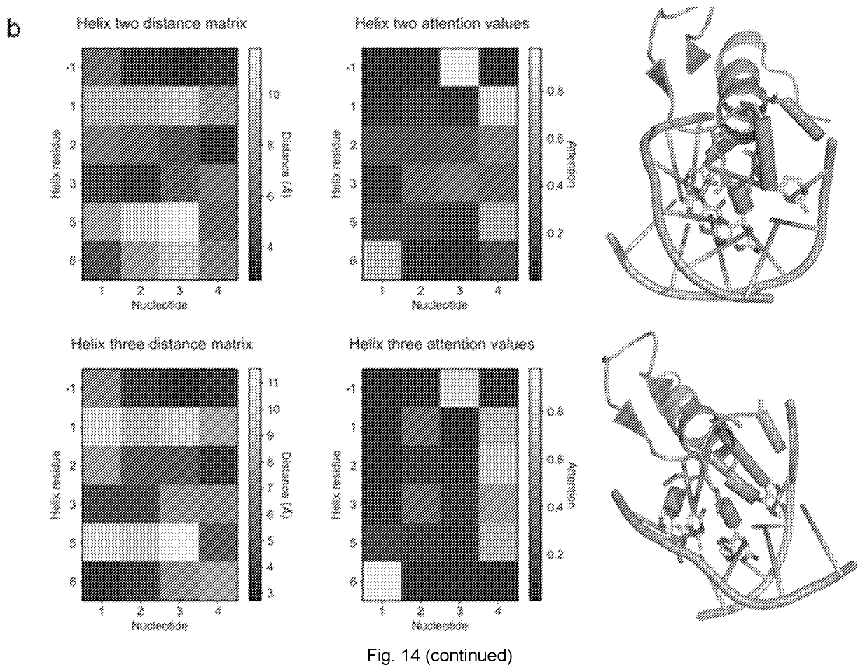

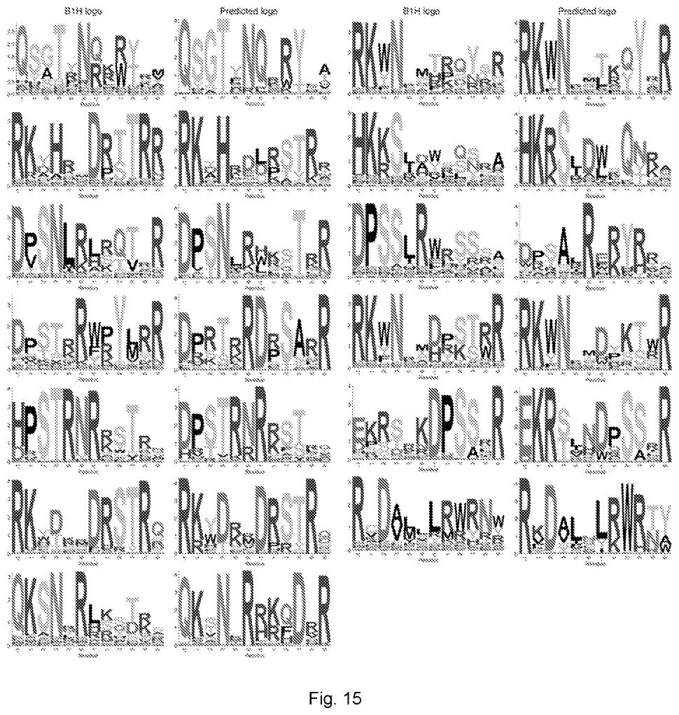

The first layer of this hierarchical architecture contains two modules that are trained on the single-finger selection data sampling a wide range of influences at the interface where adjacent finger specificity can overlap (FIG. 3A). The single-helix modules generalize to unseen sequences; residue-nucleotide relationships are captured in the attention values (FIG. 13, 14). The residue embeddings from the bottom layers are then fed into a top module which is trained on the two-helix selection data (FIG. 3B). This is akin to the experimental procedure of taking the selection pools from the single finger selections and performing two-finger selections on them. In effect, the bottom modules design functional single ZFs (for a given neighbor environment), while the top module assembles compatible ZF pairs.

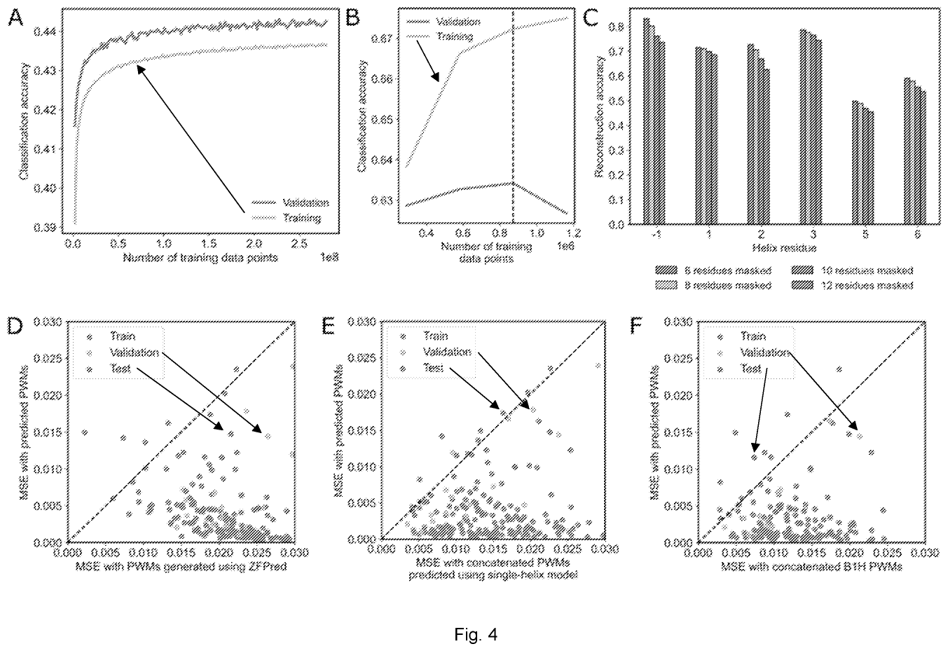

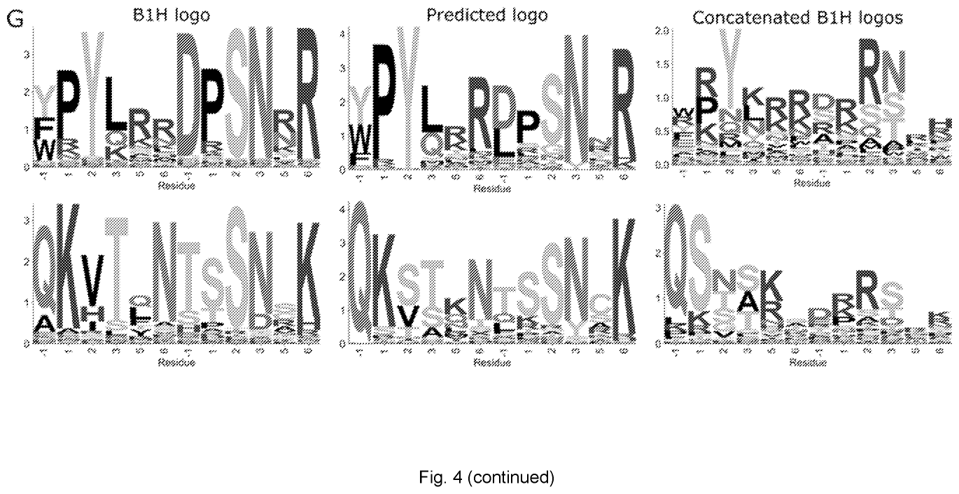

The overall model retains a traditional encoder-decoder architecture: An encoder generates a high-dimensional representation for each DNA base, a decoder then generates predictions for each residue in a ZF helix using self-attention layers and attention layers that relate the nucleotide bases to the helical residues. To train the model, we provide the nucleotide target as well as a partially masked ZF sequence and evaluate the cross-entropy loss given input data. We achieve a reconstruction accuracy (sequence identity to the six masked residues) of 0.62 and 0.69 on the validation and test data respectively; some positions (such as “−1”) that are strong determinants of binding specificity having higher reconstruction accuracies (FIG. 4A-C). Overall, as some variability in the 12 residues is allowable while retaining the ability to bind a target sequence, 0.62-0.69 reconstruction accuracy can be considered quite high (See FIG. 4C).

### ZFDesign Accurately Captures Two Helix ZF Specificity

The described method (referred to herein as ZFDesign) generates sequences in an incremental fashion: Starting from an empty sequence, the model is run once for each amino acid in the ZF helix pair. At each iteration an amino acid is predicted and this prediction is provided as context in subsequent iterations. For optimal sequence generation we adapted both an A*-based sampling methodology⁽³⁶⁾, as well as a temperature-dependent sampling procedure⁽³⁷⁾. We sought to compare ZFDesign to a baseline, but it is believed no previous model has explicitly attempted to perform full ZF-array design for a given target, with only a few collections of ZFs available. We used ZFpred, a recently developed method that outperformed previous models⁽³⁵⁾. We then used both ZFDesign and ZFpred to generate ZF sequences to target 6-mers from our test dataset. As alternative baseline comparisons, we first used the single-finger models (e.g., only the bottom module in FIG. 3B) to generate ZF sequences for each DNA 3-mer and concatenated them. In a similar fashion, we also took sequences directly from each 3-mer B1H selection and concatenated them, which is akin to previous methods of simply concatenating pre-existing collections of fingers as modules. All these three methods performed noticeably worse than our hierarchical model (See FIG. 4D-F). When directly comparing representative sequence logos of the sequences generated, ZFDesign produces logos that broadly capture the ones from the B1H two-helix selections, whereas the concatenated logos from the one-helix selections are noticeably different (See FIG. 4G, 15), underlining the fact that ZFDesign captures inter-helix relationships that are absent from the single-helix selections.

### ZFDesign, Zinc Finger Nucleases and Genomic Labeling

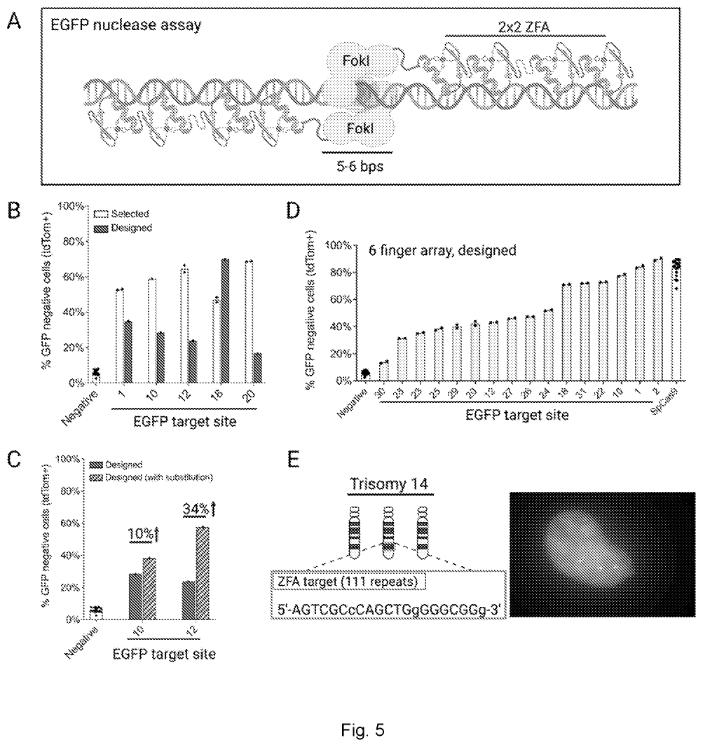

To validate ZFDesign we used a GFP-disruption assay in a U20S cell line that has been used to approximate nuclease activity for ZFNs⁽³⁸⁾, TALENs⁽³⁹⁾, and spCas9⁽⁴⁰⁾ as indels in the coding sequence of GFP lead to frameshifts and loss of fluorescence. For each ZFN, two ZF arrays were designed as ZFNs require dimerization of the Fok1catalytic domain presented as C-terminal fusions from each ZF array in a tail-to-tail orientation (FIG. 5A). The arrays use a longer linker between two-finger modules to enable independent binding as the linker allows a base to be skipped between the binding sites for each two-finger module⁽⁴¹⁾. The DNA targets for the two-finger selections detailed above had been specifically chosen to accommodate targets in the GFP coding sequence. Therefore, for each target we first assembled ZFNs that use 4ZFs per monomer (8 per ZFN) based on the most frequent pairs recovered in the corresponding 2-finger selections. Next, we designed 5 ZFNs that also use 4 ZFs per monomer to compare to the B1H selected ZFs that bind the same targets. All of the designed ZFNs are functional above background but 4 of the 5 demonstrated decreased activity relative to the selected arrays (FIG. 5B). However, the substitution of single modules can significantly increase activity (FIG. 5C) demonstrating the stringency of the assay as a single weak module can have a large impact on the overall function. Nevertheless, as these designs were functional on all targets, and longer arrays have overcome the presence of weak modules⁽⁴²⁾, we designed and tested 16 ZFNs that use 6 ZFs per monomer (12 per ZFN). We find all 16 are functional with a mean 53.6% loss of fluorescence (FIG. 5D). Finally, to determine if 6-fingers are sufficient for monomeric binding, we designed a 6-finger arrays to label a genomic locus as a GFP fusion. Many copies of GFP are necessary to visualize punctate GFP expression, so we designed the array to bind a repetitive sequence on chromosome 14, which appears in trisomy in Hek293T cells. We see 3 points of GFP by live cell imaging (FIG. 5E). These results suggest that ZFdesign consistently produces highly functional ZF arrays and that 6 or more fingers routinely produce strong on-target activity in the human genome.

### Seamless Reprogramming of Human Transcription Factors

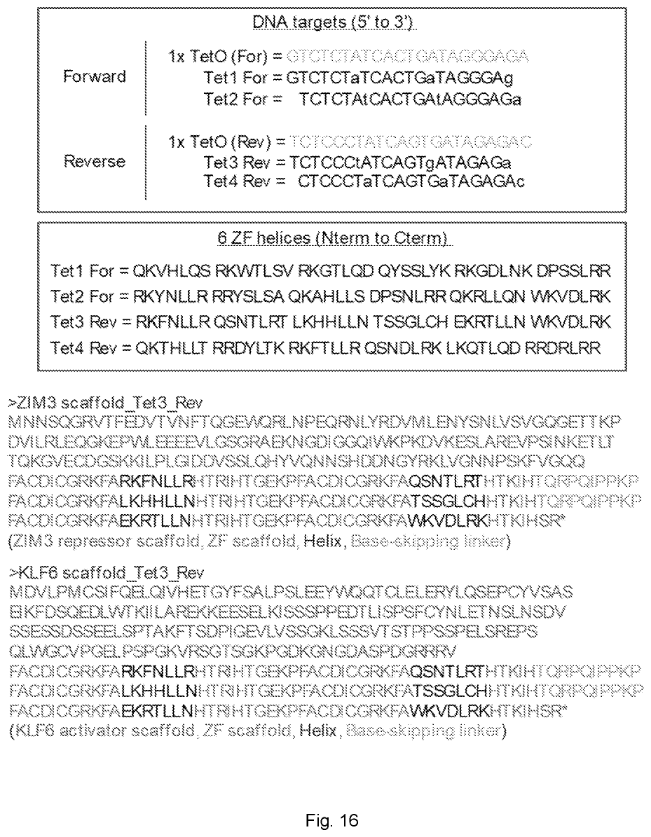

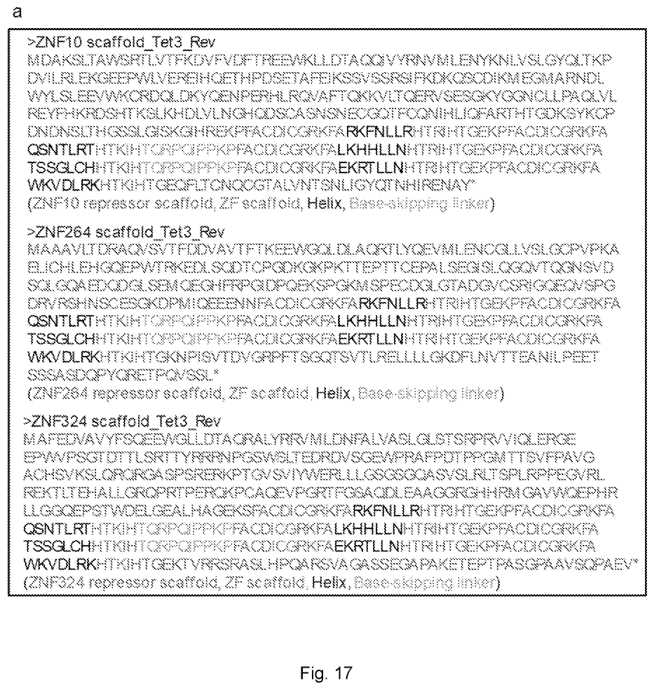

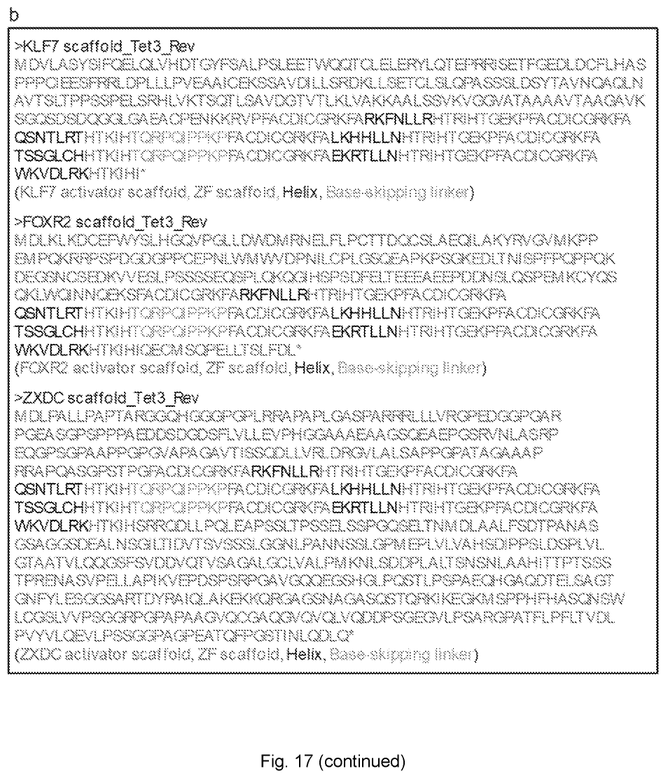

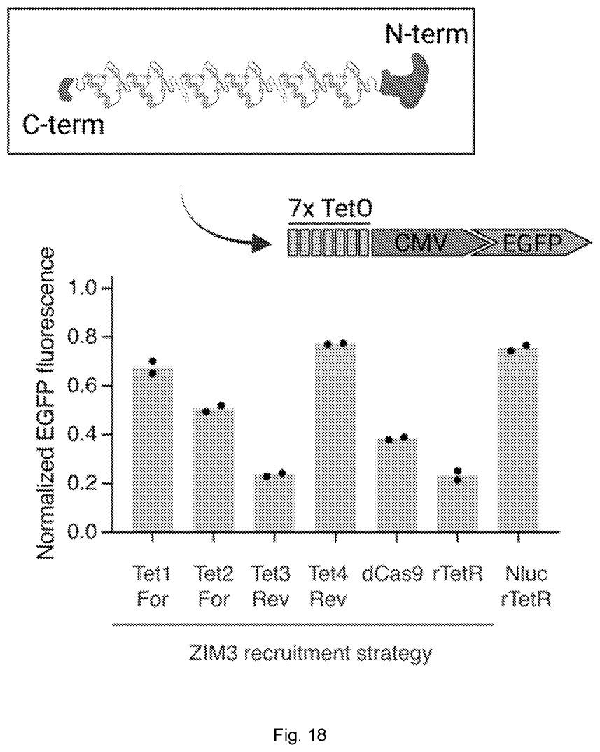

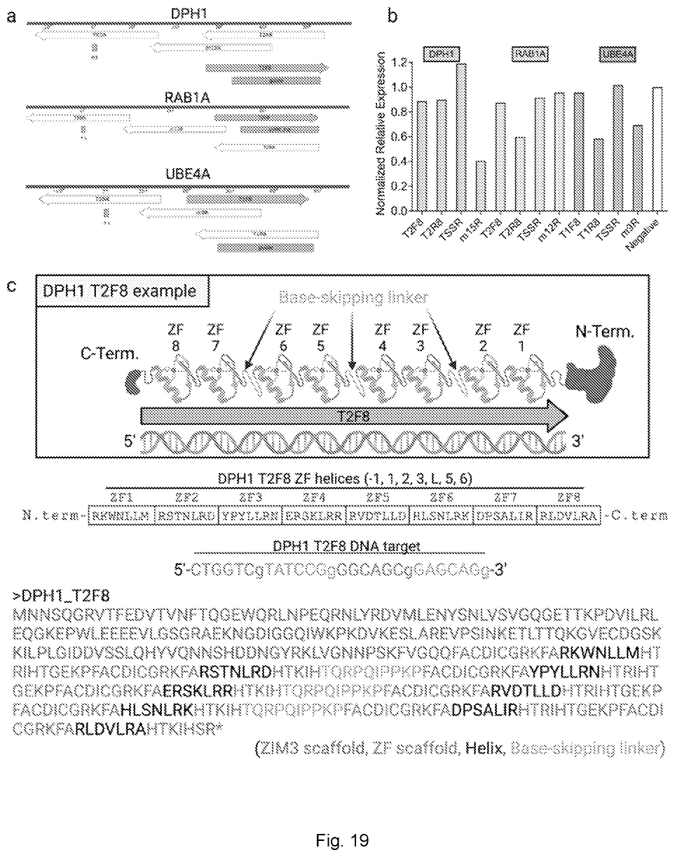

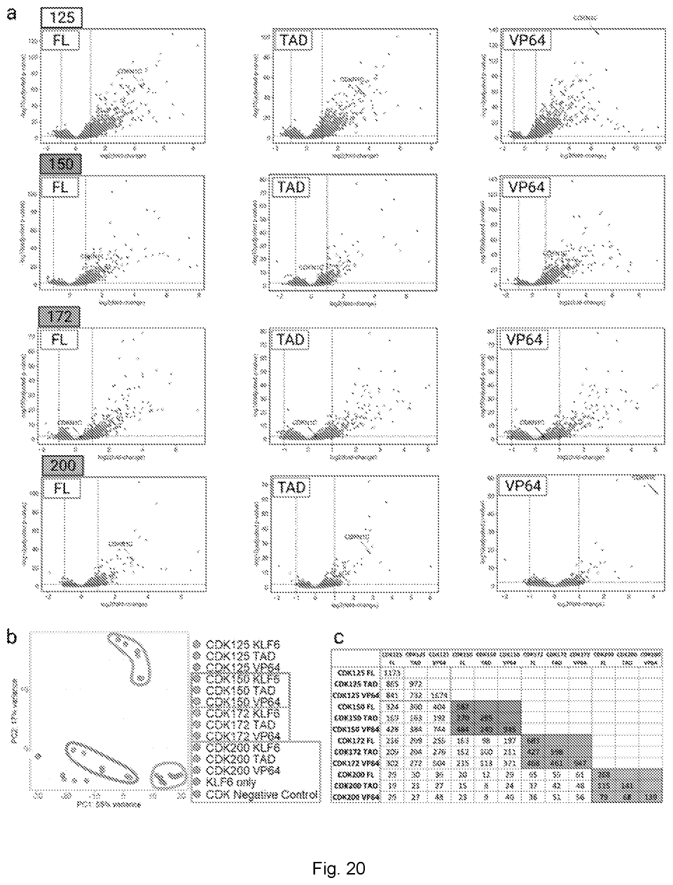

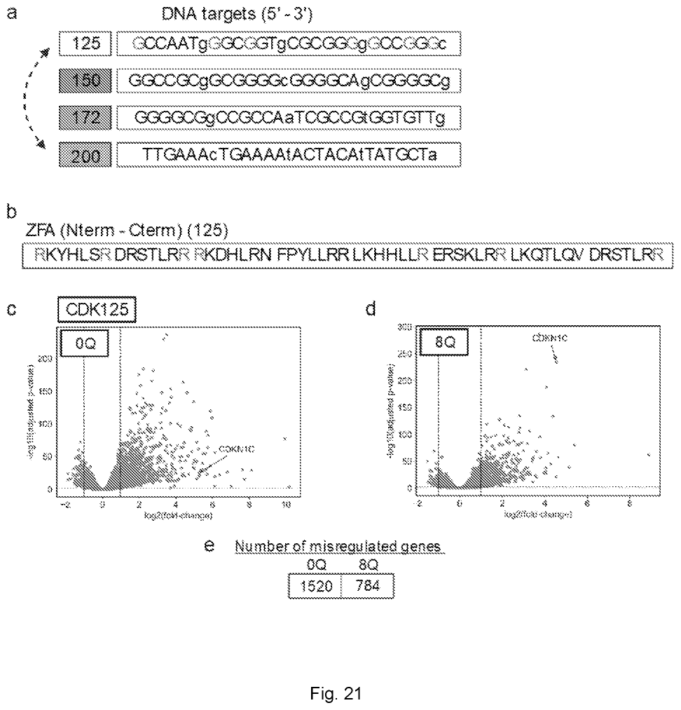

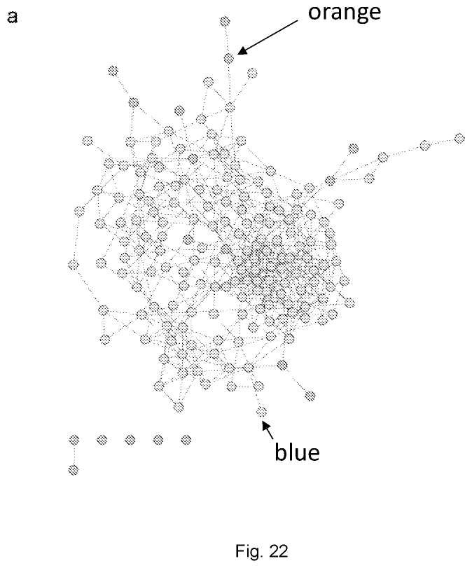

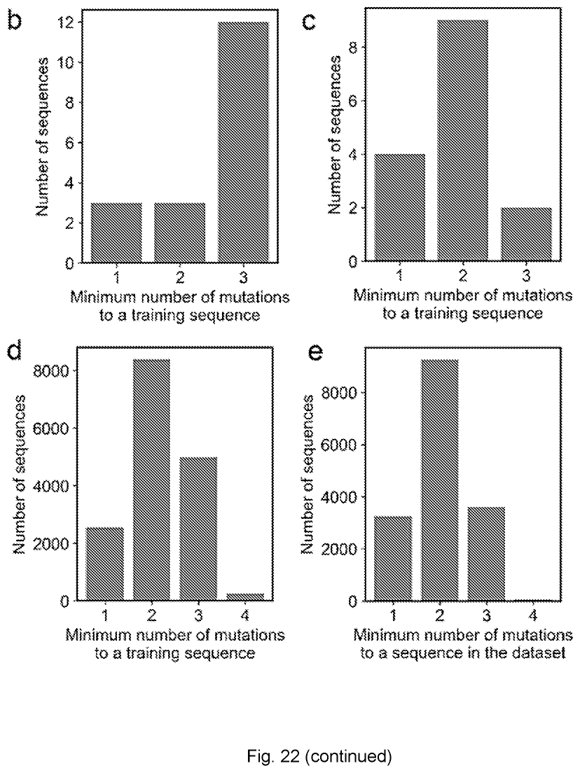

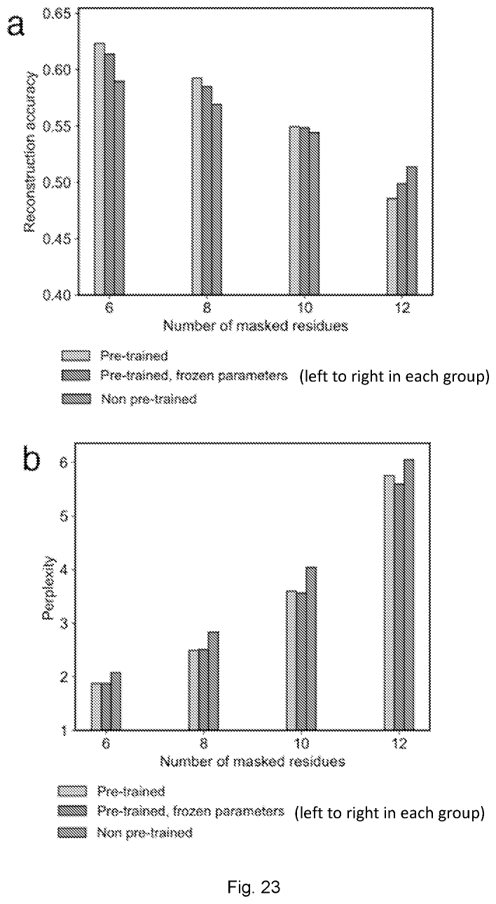

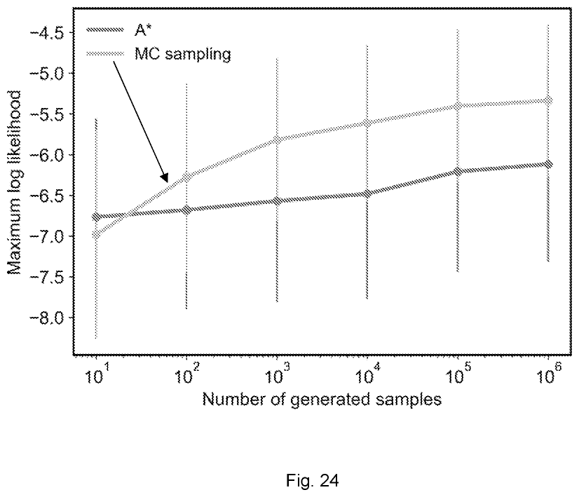

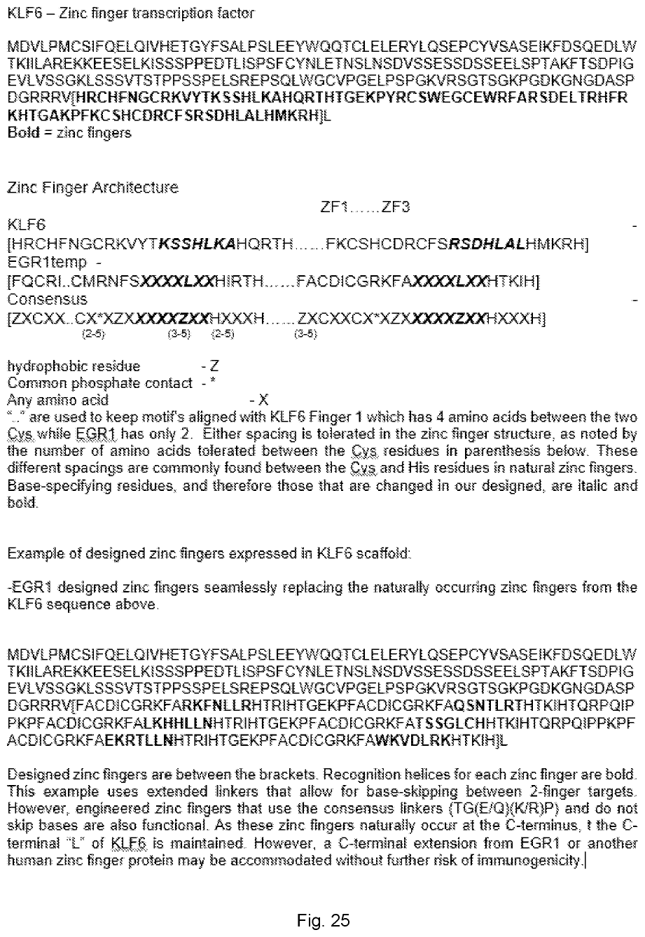

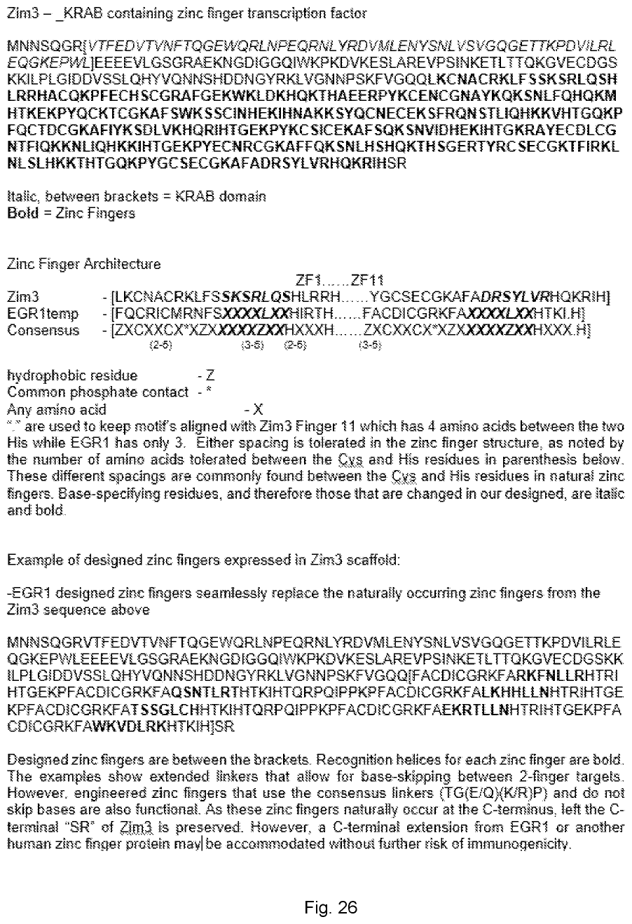

To avoid the presentation of effector domains out of their natural context, the disclosure demonstrates that ZF domains in human TFs can be seamlessly replaced with designed ZFs. This approach presents the designed ZFs in the exact context that ZFs would occur naturally in the parent protein. Such Reprogrammed Transcription Factors (RTFs) maximize secondary interactions of the TF, avoid the use of foreign effector domains, and enable investigation of TF binding events (FIG. 6A). As potential therapeutics they present maximally native-like human proteins with correspondingly low immunogenicity risk. We chose KLF6 as our activation scaffold. To test the activity of the KLF6 architecture we designed four ZF arrays to bind the TetO sequence on either the forward or reverse strand (FIG. 16). We seamlessly replaced KLF6′s ZFs with these designed ZF arrays and expressed these RTFs in a HEK293T reporter cell line that drives GFP expression with a minimal promoter (FIG. 6B). Three of the four designs activate at a similar or greater level than rTetR-VP64 with one array nearly tripling the activation level. To confirm that this RTF approach for activation was not restricted to the KLF6 protein, we replaced the DBDs of 3 other activating TFs (KLF7, FoxR2, and ZXDC) with the Tet3 ZF array (FIG. 6C). All of these RTFs activate the reporter as well or better than the rTetR-VP64 control including the FoxR2 RTF where its natural forkhead DBD was replaced with the ZF array (FIG. 17).

To create RTFs that repress target genes we used ZIM3 as our TF scaffold as ZIM3′s KRAB domain has proven a potent repressor as an isolated SpCas9 fusion⁽⁴³⁾. We replaced ZIM3′s ZFs with the series of ZF arrays designed to bind the TetO sequence as described for KLF6 (FIG. 17). We expressed these ZIM3 RTFs in a HEK293T cell line with a GFP reporter driven by a constitutive promoter. Three of the four ZF arrays repress GFP expression relative to controls with the Tet3 array out performing dCas9 (FIG. 18). Next, we replaced the ZFs of three other KRAB-containing proteins (ZNF10, ZNF264, and ZNF324) with the Tet3 ZF array. In all cases we see similar levels of repression (FIG. 6D). Interestingly, the Kox1 KRAB domain (ZNF10) provides less repression potential than the Zim3 KRAB domain when expressed as an isolated spCas9 fusion domain (43) but their activity is similar when expressed here as RTFs, suggesting that the presentation context can have a large impact on the potency of these domains.

For any of the RTFs listed above, to seamlessly replace their DBDs without impacting any other part of the parent protein, we use the consensus definition of the DBD of the parent protein to determine which part of the parent protein to replace. For example, the consensus Cys2His2 zinc finger domain begins 2 amino acids before the first Cysteine and ends with the 2^{nd} Histidine. Therefore, we replaced the natural ZFs of a TF such as Zim3, that has 11 ZFs naturally, by starting 2 amino acids before the first Cysteine of the first finger and replaced the sequence all the way through the 2^{nd} histidine in the last (eleventh) finger. This is replaced with a designed ZF array that again begins 2 amino acids before the first Cysteine of the first ZF and follows through to the 2^{nd} histidine of the last ZF in the array. No other modifications are made to the parent protein (See FIGS. 16 and 17 for exact fusion points used). For FoxR2, which uses a forkhead DBD to engage the DNA, we used the PFAM definition of the forkhead domain to precisely remove the DBD and seamlessly replace it with a ZF array, again starting 2 amino acids before the first Cysteine and ending with the 2^{nd} histidine of the last finger in the array.

Representative constructs are shown on FIG. 25, showing a KLF6-Zinc finger transcription factor, and FIG. 25, showing a Zim3-_KRAB containing zinc finger transcription factor.

MDVLPMCSIFQELQIVHETGYFSALPSLEEYWQQTCLELERYLQSEPCY
VSASEIKFDSQEDLWTKIILAREKKEESELKISSSPPEDTLISPSFCYN
LETNSLNSDVSSESSDSSEELSPTAKFTSDPIGEVLVSSGKLSSSVTST
PPSSPELSREPSQLWGCVPGELPSPGKVRSGTSGKPGDKGNGDASPDGR
RRV[HRCHFNGCRKVYTKSSHLKAHQRTHTGEKPYRCSWEGCEWRFARS
DELTRHFRKHTGAKPFKCSHCDRCFSRSDHLALHMKRH]L

Bold=zinc fingers

### Zinc Finger Architecture

```
                                                                  ZF1... ...ZF3
KLF6 -
[HRCHFNGCRKVYT [symbol] HQRTH... ...FKCSHCDRCFS [symbol] HMKRH]
EGR1temp-
[FQCRI..CMRNFS [symbol] HIRTH... ...FACDICGRKFA [symbol] HTKIH]
Consensus -
[ZXCXX..CX*ZXZ [symbol] HXXXH... ...ZXCXXCX*XZX [symbol] HXXXH]
                (2-5) (3-5) (2-5) (3-5)
```

hydrophobic residue-Z
Common phosphate contact-*
Any amino acid-X
“. . . ” are used to keep motif's aligned with KLF6 Finger 1 which has 4 amino acids between the two Cys while EGR1 has only 2. Either spacing is tolerated in the zinc finger structure, as noted by the number of amino acids tolerated between the Cys residues in parenthesis below. These different spacings are commonly found between the Cys and His residues in natural zinc fingers. Base-specifying residues, and therefore those that are changed in our designed, are italic and bold.

Example of designed zinc fingers expressed in KLF6 scaffold:
EGR1 designed zinc fingers seamlessly replacing the naturally occurring zinc fingers from the KLF6 sequence above.

MDVLPMCSIFQELQIVHETGYFSALPSLEEYWQQTCLELERYLQSEPCY
VSASEIKFDSQEDLWTKIILAREKKEESELKISSSPPEDTLISPSFCYN
LETNSLNSDVSSESSDSSEELSPTAKFTSDPIGEVLVSSGKLSSSVTST
PPSSPELSREPSQLWGCVPGELPSPGKVRSGTSGKPGDKGNGDASPDGR
RRV[FACDICGRKFARKFNLLRHTRIHTGEKPFACDICGRKFAQSNTLR
THTKIHTQRPQIPPKPFACDICGRKFALKHHLLNHTRIHTGEKPFACDI
CGRKFATSSGLCHHTKIHTQRPQIPPKPFACDICGRKFAEKRTLLNHTR
IHTGEKPFACDICGRKFAWKVDLRKHTKIH]L

Designed zinc fingers are between the brackets. Recognition helices for each zinc finger are bold. In the example we are using extended linkers that allow for base-skipping between 2-finger targets. However, engineered zinc fingers that use the consensus linkers (TG(E/Q)(K/R)P) and do not skip bases are also functional. As these zinc fingers naturally occur at the C-terminus, we have left the C-terminal “L” of KLF6, however, a C-terminal extension from EGR1 or another human zinc finger protein may be accommodated without further risk of immunogenicity.

### Zim3-_KRAB Containing Zinc Finger Transcription Factor

MNNSQGR[VTFEDVTVNFTQGEWORLNPEQRNLYRDVMLENYSNLVSVG
QGETTKPDVILRLEQGKEPWL]EEEEVLGSGRAEKNGDIGGQIWKPKDV
KESLAREVPSINKETLTTQKGVECDGSKKILPLGIDDVSSLQHYVQNNS
HDDNGYRKLVGNNPSKFVGQQLKCNACRKLFSSKSRLQSHLRRHACQKP
FECHSCGRAFGEKWKLDKHQKTHAEERPYKCENCGNAYKQKSNLFQHQK
MHTKEKPYQCKTCGKAFSWKSSCINHEKIHNAKKSYQCNECEKSFRQNS
TLIQHKKVHTGQKPFQCTDCGKAFIYKSDLVKHQRIHTGEKPYKCSICE
KAFSQKSNVIDHEKIHTGKRAYECDLCGNTFIQKKNLIQHKKIHTGEKP
YECNRCGKAFFQKSNLHSHQKTHSGERTYRCSECGKTFIRKLNLSLHKK
THTGQKPYGCSECGKAFADRSYLVRHQKRIHSR

Italic, between brackets=KRAB domain
Bold=Zinc Fingers

### Zinc Finger Architecture

| | ZF1... ...ZF11 |
|---|---|
| Zim3 | -[LKCNACRKLFS [symbol] HLRRH... ...YGCSECGKAFA [symbol] HQKRIH] |
| EGR1temp | -[FQCRICMRNFS [symbol] HIRTH... ...FACDICGRKFA [symbol] HTKI.H] |
| Consensus | -[ZXCXXCX*XZX [symbol] HXXXH... ...ZXCXXCX*XZX [symbol] HXXX.H] |
| | (2-5) (3-5) (2-5) (3-5) |

hydrophobic residue-Z
Common phosphate contact-*
Any amino acid-X
“. . . ” are used to keep motif's aligned with Zim3 Finger 11 which has 4 amino acids between the two His while EGR1 has only 3. Either spacing is tolerated in the zinc finger structure, as noted by the number of amino acids tolerated between the Cys and His residues in parenthesis below. These different spacings are commonly found between the Cys and His residues in natural zinc fingers. Base-specifying residues, and therefore those that are changed in our designed, are italic and bold.

Example of designed zinc fingers expressed in Zim3 scaffold:
EGR1 designed zinc fingers seamlessly replace the naturally occurring zinc fingers from the Zim3 sequence above

MNNSQGRVTFEDVTVNFTQGEWQRLNPEQRNLYRDVMLENYSNLVSVGQ
GETTKPDVILRLEQGKEPWLEEEEVLGSGRAEKNGDIGGQIWKPKDVKE
SLAREVPSINKETLTTQKGVECDGSKKILPLGIDDVSSLQHYVQNNSHD
DNGYRKLVGNNPSKFVGQQ[FACDICGRKFARKFNLLRHTRIHTGEKPF
ACDICGRKFAQSNTLRTHTKIHTQRPQIPPKPFACDICGRKFALKHHLL
NHTRIHTGEKPFACDICGRKFATSSGLCHHTKIHTQRPQIPPKPFACDI
CGRKFAEKRTLLNHTRIHTGEKPFACDICGRKFAWKVDLRKHTKIH]SR

Designed zinc fingers are between the brackets. Recognition helices for each zinc finger are bold. In the example we are using extended linkers that allow for base-skipping between 2-finger targets. However, engineered zinc fingers that use the consensus linkers (TG(E/Q)(K/R)P) and do not skip bases are also functional. As these zinc fingers naturally occur at the C-terminus, we have left the C-terminal “SR” of Zim3, however, a C-terminal extension from EGR1 or another human zinc finger protein may be accommodated without further risk of immunogenicity.

To test the regulatory potential of endogenous genes with RTFs, we applied the ZIM3 architecture to repress 3 endogenous targets (DPH1, Rab1a, and UEB4A) and designed 4 arrays each to bind sequences close to the transcriptional start site (TSS) of each gene. To maximize the likelihood of function, we designed these and all following ZF arrays to use 8-fingers. HEK293T's were nucleofected with the RTFs and expression levels assayed by RT-qPCR. For each target gene at least one construct reduced expression levels significantly (FIGS. 6E and 19). To activate an endogenous target, we reprogrammed KLF6 with a series of arrays designed to bind a 150 bp region upstream of the TSS in the CDKN1C promoter. All 7 RTFs increased the expression of CDKN1C with 3 of the 7 by 9 to 43-fold (FIG. 6F).

### Genome-wide Regulatory Activity of Reprogrammed Transcription Factors

ZFDesign enables the reprogramming of TFs for either activation or repression. To test the precision of the regulation we used RNA-seq to quantify the on and off-target regulation of the RTFs. We focused on the 4 most potent KLF6 RTF regulators of CDKN1C, #125, 150, 172, and 200 (see FIG. 6F). In all cases but #172 we found that CDKN1C was one of the most upregulated genes (FIG. 20). However, between 268 to 1173 off-target genes were also activated. Since KLF6 is a human TF, we analyzed whether off-target activity is due to secondary interactions of the TF and not the ZF arrays. We thus tested KLF6 without any ZFs as well as the 4 ZF arrays as full KLF6 RTFs, as fusions with the KLF6 truncated transactivation domain, and as fusions with VP64. RNA-seq on each of these constructs indicates that off-target activity is primarily dictated by the ZF arrays (FIG. 20).

The specificity of ZF arrays can be impacted by target content and affinity. As noted, G-rich binding tends to be more promiscuous. Consistent with this observation, the CDKN1C target with the lowest G-content (#200, FIG. 21) also led to the least number of off-target events. In addition to minimizing target G-content, ZF specificity can be improved by reducing the nonspecific affinity provided by contacts made between each ZF and the phosphate backbone^{(44, 45)} (FIG. 6G). This puts more pressure on the base-specifying interaction of each helix to provide the binding affinity necessary for function. We created mutant versions of CDKNIC RTF #200 that replace either 2, 4, or 8 of the phosphate-contacting arginines with glutamines. We first compared the impact of these mutations by qPCR both on-target and at two off-target loci upregulated in the RNA-seq screens (FIG. 6F, right). The expression of these off-target genes is reduced by up to 70 or 55%, respectively, as we increase the number of phosphate-contacting modifications while the on-target activity is only reduced by 12%. Next, RNA-seq demonstrates the number of off-targets is decreased with the number of modifications and that only CDKN1C is upregulated with the full 8 arginine to glutamine modifications, thus providing single target resolution. Taking the same approach with the G-rich binding #125 cut the number of off-targets in half but elimination of off-target activity will likely require the design of ZF-arrays that use alternative binding strategies for G-rich targets (FIG. 21).

It will be recognized from the foregoing Examples this disclosure presents ZFDesign, a novel hierarchical attention-based AI model trained on comprehensive screens of ZF-DNA interactions that consider the influence of multiple adjacent finger environments. ZFDesign captures these influences to provide the first general design model for ZF arrays. By contrast, previous efforts produced incomplete collections of ZF modules that often fail out of context and produce low on-target activity. Conversely, the described model consistently produced ZF arrays across a wide range of targets with high efficacy as nucleases, repressors, and activators. Thus, ZFDesign represents a significant advance as the design of ZFs for any given target is suitable for study of many research and therapeutic applications with the advantages of small size and low immunogenicity.

Without intending to be constrained by any particular theory, it is considered the disclosure provides the first generalizable design methodology that allows for the seamless replacement of a TF's natural DNA-binding domain and direct the TF to any target of interest. These RTFs can produce activation and repression activities similar to CRISPR-based tools, supporting utility of these proteins as therapeutics comprised of solely human components. In addition, the described approaches all for analyzing TF function as they more accurately mimic natural TFs.

The following materials and methods were used to produce the data described herein and in the accompanying figures.

### Library Builds

Primary zinc finger libraries: All primary ZF libraries were built as previous described^{(35, 46)} and detailed below. To provide templates for PCR, gBlocks were ordered from IDT that coded for the finger 0 and finger 1 domains of each library (FIG. 9, and see FIG. 1 for numbering of domains). The critical differences that distinguish each library from one another is that they each place a different environment at the interface between domain 1 and the library domain 2. These libraries include five domain 1 interactions that bind A at the interface, five that bind C at the interface, and two that bind G. These libraries use side chains at the interface with a range of biochemical properties to interact with the overlap base (basic, acidic, polar, aromatic, and hydrophobic interactions). Together, the biochemical property of the side chain at position 6 of domain 1 and the base it specifies at the overlap position represent the unique interface environment offered by each library. Next, an oligonucleotide was design with degeneracy (NNS) at the codon positions corresponding to the six critical residue positions of the ZF domain 2 alpha helix. This oligo was used for all library builds, only the template gBlock, and therefore the 0 and 1 domains, are changed. PCR was used to generate the library insert, amplifying from the library-specific gBlock template with the library oligonucleotide paired with a downstream oligonucleotide used to capture the full 3-finger insert. For each library, PCR reactions were run in 96-well plate format and pooled. The PCR products were digested with Kpn1 and XbaI and ligated into 15 μg of digested B1H expression vector. Ligations were run over night at 16° C., ethanol precipitated, and resuspended in 15 μl of 10 mM Tris-Cl, pH 8.5. The ligation was electroporated into 15 aliquotes of electrocompetent US0 cells and recovered in 1 L of SOC. One-hour post electroporation, 200 μl of the culture was titered in 10-fold serial dilution on Carbenicillin plates to determine library size. To select for transformants, carbenicillin was then added to the culture at this point and grown to mid-log. The library DNA was then recovered by Qiagen maxiprep. Library sizes ranged from 1-3×10⁹. This approach has been shown to consistently produce libraries with diversities that approximate random⁽⁴⁶⁾.

2-finger libraries: Second round selections were used to select compatible pairs from pre-selected ZF pools generated in the primary ZF library selections. We pooled recovered plasmid DNA from our primary single-finger screens on a binding site basis, resulting in a pool of diverse helices (termed “round 2 pools”) with broad compatibility for each of the 64 different binding sites. To ensure these were enriched for functional helices and not background, a simple cutoff was devised to omit unsuccessful selections. Based on the data filtering metrics described, single-finger pools were omitted if less than 20% of the reads passed these filters as those selections would have added a disproportionate amount of non-functional ZFs to our template pools. This set of 64, round 2 pools was used as a PCR template to create either ‘domain 1’ or ‘domain 2’ amplicons using Expand™ High Fidelity PCR system (Roche) and 15 cycles of PCR to reduce bias. ‘domain 1’ and ‘domain 2’ reactions were gel-purified from a 2% agarose gel, quantified by nanodrop, and stored at −20 C. In order to create a 2-finger library insert, we performed overlapping PCR to stitch appropriate ‘domain 1’ and ‘domain 2’ pools together. Purified single-finger amplicons were combined equimolar as the template for overlap PCR with Phusion® High Fidelity DNA Polymerase (NEB) (25 cycles), PCR-purified, digested with KpnI and NotI, gel-purified, and quantified by Nanodrop (ThermoFisher Scientific). The digested 2-finger library inserts were ligated into our 2-finger library vector (see FIG. 2D). Ligations were performed overnight at 16 C using 300 ng of digested backbone and a 5:1 molar excess insert:backbone. Ligations were ethanol precipitated and resuspended in 5 uL EB (Qiagen). 100 ng of the ligation was electroporated into USO-ω) cells, recovered in SOC for 1 hr, titered on 2xYT agar plates containing 2% glucose and 100 ug/mL carbenicillin, and stored at 4 C overnight. Based on cell counts the following day, 5×10⁶ cells were plated on 15 cm rich media agar plates (2xYT, 2% glucose, 100 ug/mL carbenicillin), grown at 30 C for 12-14 hours, harvested by scraping, and finally miniprepped to obtain final round 2 libraries.

### Zinc Finger Selections

Primary ZF Libraries: Libraries were built in a vector that will express the ZFs as a fusion to the omega subunit of the bacterial polymerase using a strong promoter. In the B1H system omega is simply acting as an activation domain. The binding site reporter vectors were built by placing the binding site of interest 10 bp upstream of the-35 box of the promoter that drives HIS3 and GFP expression in the previously described GHUC vector. For example, for the library 2 TAC selection, the binding site 5′ TAC-ACA-AAG 3′ was built into the GHUC vector 10 bp upstream of the promoter where the library domain will bind TAC and domains 1 and 0 of library 2 will bind ACA and AAG, respectively (FIG. 1C). For each selection, the ΔrpoZ selection strain was transformed with the ZF library and the appropriate reporter plasmid by electroporation. The cells were expanded in 10 ml SOC for 1 h at 37 C with rotation, recovered and resuspended in minimal media supplemented with histidine and grown with rotation for an additional hour at 37 C. Finally, cells were washed in minimal media that lacks histidine, recovered in 1 ml of this media, and 20 μl's plated in serial dilution on rich plates containing Kanamycin and Carbenicillin to quantify double transformants. This plate was grown at 37 C overnight while the remaining 980 μl of transformed cells was stored at 4 C. Once grown, the serial dilutions were counted and a volume containing a minimum of 5×10⁸ cells were taken from the transformants stored at 4 C and plated on selective media. These plates contained 2 mM 3-AT, a competitive inhibitor of HIS3, that helps to removed background activity from the screen. Cells were grown on the selection plates for 36-48 h at 37 C. Colonies were counted, cells were pooled, and DNA harvested. This DNA was used as the template for Illumina sequencing. All selections resulted in hundreds to thousands of surviving colonies.

Compatible 2-finger modules selections: In order to identify compatible 2-finger modules from our round 2 libraries, we first built a matching set of vectors containing the intended DNA target and then leveraged omega-dependent activation of the HIS3 reporter in our bacteria 1-hybrid system. Round 2 libraries were co-transformed with the matching reporter vector in USO-ω cells and recovered and titered as described. Based on cell counts the next day, 1×10⁶ cells were added in triplicate to a 96-well deep-well plate containing a sterile bead for efficient agitation. Selections were performed in 1 mL NM+Ura/−His supplemented with 100 μg/mL carbenicillin, 50 μg/mL Kanamycin, 1 μM IPTG, and 5 mM 3AT. These were grown at 37 C in a plate shaker for 18, 24, or 40 hours and harvested upon reaching visible turbidity (typically OD>0.6). Triplicates were pooled, miniprepped, and deep sequenced on an Illumina NextSeq 500. Helices were rank-ordered by sequencing reads, and 2-finger modules within the top 5 highest counts were chosen for follow-up assembly and testing in the EGFP nuclease assay.

### U2OS GFP Disruption Assay

Zinc finger nuclease (ZFN) activity was assessed by measuring disruption of an integrated, constitutively-expressed eGFP reporter in a clonal U2OS cell line previously described⁽³⁹⁾. Cells were cultured in DMEM supplemented with 10% FBS, 2 mM GlutaMAX™ (Life Technologies), 1% penicillin/streptomycin, 1% MEM non-essential amino acids (Life Technologies), 2 mM sodium pyruvate, and 400 μm/mL G418. 1 μg of each ZFN monomer plasmid DNA and 200 ng ptdTomato-N1 plasmid DNA were transfected in duplicate into 5×10⁵ cells using a Lonza Nucleofector™ 2b Device (Kit V, Program X-001). In each assay 2 μg of the parental empty vector (a modified derivative of the JDS71 vector from addgene) and 200 ng ptdTomato-N1 was used as a negative control, and 2 μg of a dual spCas9-guide expressing vector (modified addgene plasmid #41815) and 200 ng ptdTomato-N1 was used as a positive control in each experiment. Cells were grown in 6-well dishes for 3 days post-transfection, harvested and kept on ice, and analyzed for expression of eGFP and tdTomato on a Sony SH800 cell sorter. In order to restrict analysis to only cells that likely received both ZFN monomer plasmids, populations were first gated on the top 15-25% tdTomato+cells, and then analyzed for loss of eGFP expression.

### Next Generation Sequencing and Prep

Primary libraries: Following selection from ≥5×10⁸ library variants, surviving colonies were pooled, miniprepped, and DNA barcoded for sequencing on an Illumina NextSeq® 500. Typically these were performed as a set of 64 3 bp binding sites for a given ‘overlap’ library as follows. 2 uL of pooled plasmid DNA was used as a template for barcoding in a 25 μL reaction with Taq Polymerase (NEB) with the following cycling parameters: 95 C for 5 min, 20 cycles of [95 C:20 s, 52 C:30 s, 68 C:30 s], 68 C for 10 min, and held at 4 C. 5 μL each reaction was visualized on a 1% agarose gel to confirm apparent equal amplification. All 64 reactions were pooled in equal volumes. These were run out on a 1% agarose gel, gel purified, and submitted to the NYU Genome Technology Center for sequencing on a NextSeq® 500.

2-finger libraries: Following selection of ˜3×10⁶ 2F library variants, plasmid DNA was extracted from surviving cells and barcoded for deep sequencing on an Illumina NextSeq® 500 as follows. 24, pooled plasmid DNA was used as a template for barcoding in a 25 μL reaction with GoTaq® Green 2X Mastermix (Promega) with the following cycling conditions: 95 C for 5 min, 15 cycles of [95 C:30 s, 68 C:30 s, 72 C:60 s], 72 C for 5 min, and held at 4 C. 10 μL each reaction was visualized on a 1% agarose gel to confirm equal amplification, all reactions were pooled in equal volumes. These were gel-purified from a 1% agarose gel, and submitted to the NYU Genome Technology Center for sequencing on an Illumina NextSeq® 500.

### Sequence Recovery and Filtering

All paired end Illumina reads are demultiplexed and trimmed into 21-mers with in-house Unix scripts based on EMBOSS 6.6.0. Trimmed DNA sequences are translated, and amino acid sequences are considered if they have a least two read counts and are coded by at least two different DNAs. The invariant Leucine at the helix position +4 is excluded.

### Clustering and Filtering Selections

For each selection, helix sequences were clustered using the MUSI software⁽³⁴⁾. Each sequence was assigned to the cluster associated with the PWM for which it was assigned the highest responsibility. For each cluster generated, the Shannon entropy value was calculated for each helix residue based on the PWM for that cluster. If a selection lacked a cluster with at least one position with an entropy of two or less, that selection was filtered out for downstream analysis.

### Computing Similarity Between Selections by Hamming Distance

To compare the helices from two selections, A and B, pairwise normalized Hamming distances were computed between the two sets of filtered sequences based on the number of identical amino acids. The minimum normalized Hamming distance was then computed from each helix in selection A to each helix in selection B as well as from each helix in selection B to each helix in selection A. The overall distance between the two selections was computed as the mean of these distances.

### Molecular Dynamic Simulations

Similar to our previous studies^{(47, 48)}, the PDB file 1AAY⁽⁴⁹⁾ was used as template, the DNA was elongated by 2 bp at each end using X3DNA to avoid the melting end effect so that the binding of zinc fingers is not affected. The DNA and protein sequences were mutated using Chimera (www.cgl.ucsf.edu/chimera/) for each library and test case, the protonated states were determined by WHATIF (swift.cmbi.umcn.nl/whatif/) The prepared structures were then solvated into a TIP3P water box with 15-Å buffer of water extending from the protein/DNA complex in each direction, sodium ions were added to ensure the overall charge neutrality. The FF99 Barcelona forcefield was used for protein/DNA complex and zinc amber forcefieid for zinc ions. The particle mesh Ewald method was used for electrostatics calculations. The SHAKE algorithm was used to constrain the hydrogen-containing bond lengths, which allowed a 2-fs time step for MD simulation. The non-bonded cut-off was set to 12.0 Å. The systems were energy minimized using a combination of steepest descent and conjugate gradient methods. Then the systems were thermalized and equilibrated for 3 ns using a multistage protocol. The first step was a 1.5 ns gradual heating from 100K to 300 K, followed by 1.5 ns of density equilibration, both at 1-fs step length. Berendsen thermostat and barostat were used for both temperature and pressure regulation for another 6-ns equilibration at 2-fs step length with gradually reduced positional constraints at 300K. The systems were built with tleap and the simulations were conducted with GPU accelerated Amber18⁽⁵⁰⁾. For each system, three 500-ns trajectories were simulated. The hydrogen bond analysis was performed using BioPython. We considered as a hydrogen bonds any contacts below 3.5 Å between the atoms O6 and N7 in a Guanine and the atoms NH1 and NH2 in an Arginine or ND2 and OD1 for an Asparagine. Bifurcated hydrogen bonds between a guanine and an arginine are identified when two pairs 06-NH1/2 and N7-NH1/2 are found, allowing the tautomeric bifurcated hydrogen bond.

### Calculating Entropy of Binding for Core Helices Across Libraries

To quantify the promiscuity of helices that target each nucleotide three-mer, the Shannon entropy was computed. For each nucleotide three-mer, a position frequency matrix of nucleotide sequences targeted by every set of core residues (−1, 2, 3, 6) was computed. The entropy was calculated in a position wise fashion and then summed to get an overall metric for specificity.

### Neural Network Architecture

We developed a hierarchical neural network architecture that mimics the B1H experimental setup and captures the modularity of zinc finger proteins. This architecture is composed of three modules (FIG. 3). The first two modules are trained to generate helices that bind to a particular nucleotide four-mer which includes the target three-mer and the overlap base. The residue embeddings from these modules are concatenated and used as input to a third module that is designed to learn compatibility between the helices in a pair (FIG. 3A). The first module generates residue embeddings for the first helix in a pair based on the last four bases in a target seven-mer and the second module generates residue embeddings for the second helix based on the first four bases in a target seven-mer (FIG. 3B). The full model is trained to predict all the core residues in two helices given a nucleotide seven-mer.

The architecture of the first two modules is largely based on the Transformer model¹. An encoder generates a high-dimensional representation for each base in a nucleotide four-mer. A decoder then generates predictions for each core residue in a zinc finger helix using self-attention layers and attention layers that relate the nucleotide bases to the helix residues. While the decoder in a conventional Transformer strictly generates sequences from left to right¹, the decoders in this model use bi-directional information. A portion of the residues in a helix are masked and the decoder outputs amino acid predictions at these positions. The third module consists of repeating self-attention layers and feed forward layers that allow the model to update residue embeddings based on inter-helix compatibility (FIG. 3B).

Variants of the first module with different numbers of attention heads and embedding dimensions were trained and evaluated on the initial task of predicting residues in a single helix (Table A). In the final model, all attention layers were repeated three times and each attention layer had four heads. The model embedding dimension (d_{model}) was set to 128. The value and key embedding dimensions for computing scaled dot-product attention (dᵥ and dₖ) were both set to 256. The hidden dimension in the feed-forward layers was set to 128. For regularization, dropout layers were included after every feed forward and attention layer with a dropout percentage of 0.3.

Table A shows the number of human transcription factors that use five common DNA-binding domains⁽⁹⁾ and their comparative size. As many DNA-binding domains require dimerization, their monomeric and multimeric sizes are listed. A comparison of the multimeric size and the domain's common target length allows a calculation of amino acids required per base specified.

TABLE A

| DNA-binding | Human | Monomeric | Functional | | ~Target | aa's per |
|---|---|---|---|---|---|---|
| domain | TFs(9) | size (aa) | state | Total size (aa) | length (bp) | base |
| Forkhead | 49 | 102 | Monomer | 204 | 12 | 17 |
| | | | or dimer | | | |
| Basic Leucine | 71 | 61 | Dimer | 122 | 10 | 12.2 |
| Zipper | | | | | | |
| Basic helix- | 111 | 54 | Dimer | 108 | 8 | 13.5 |
| loop-helix | | | | | | |
| Homeodomain | 222 | 60 | Monomer | 120 | 12 | 10 |
| | | | or dimer | | | |
| C₂H₂ zinc | 760 | 28 | Monomer | 252 (The average | 3-4 bp per | 9.3 |
| finger | | | (Requires | human ZF-TF | monomer | |
| | | | arrays of | has 9 ZFs) | | |
| | | | multiple | | | |
| | | | domains) | | | |
| SpCas9 | — | 1368 | Monomer | 1368 | 23 | 59.4 |

### Training Datasets

The models were trained and evaluated on data derived from B1H selections. B1H screening data was filtered using a previously described approach, where helices were evaluated based on the diversity of encoding nucleotide sequences found in the screen²⁻⁴. The Shannon entropy for each helix (or helix pair) was calculated based on the number of reads associated with each possible encoding nucleotide sequence. Helices were filtered based on previously defined thresholds³. Specifically, helices with less than ten reads or a Shannon entropy of less than 0.07 were removed.

Modules one and two were pre-trained using data from single-helix B1H selections that were performed against nucleotide four-mers. The data included selections performed with 11 libraries against 192 different nucleotide four-mers. In total, the dataset included 2,071,764 data points. For initial training and hyperparameter tuning, the data points were split into train, test, and validation datasets at proportions of 80%, 10%, and 10% respectively by four-mer sequence. For pre-training, the data was instead split by helix sequence.

The full model was trained using data from helix-pair B1H selections that were performed against nucleotide seven-mers. An initial dataset of selections against 189 seven-mers was split into training and validation datasets at proportions of 90% and 10%. This dataset contains a total of 327,792 data points. To ensure that the validation set was sufficiently different from the training dataset, a graph was generated where nucleotide seven-mers were represented as nodes and edges connected seven-mers within two base substitutions from each other. While most of the nodes formed a single connected component, there were separate components that were included in the validation dataset (FIG. 22A). Nodes with the lowest degree in the graph, and their neighbors, were then added to the validation dataset. Most of the sequences in the validation dataset were consequently at least three mutations away from any sequence in the training dataset (FIG. 22B). A separate set of 15 selections filtered to ensure at least 100 unique helix pairs was used as an independent test set for model evaluation.

### Model Training

In both training steps, a nucleotide target and a sequence of partially masked core residues from either a single zinc finger or a helix pair were provided to the model. 50% of the core residues were masked and the cross-entropy loss was evaluated based on the output probabilities. Training was done using an Adam optimizer with a learning rate of 1e-4, and a minibatch size of 128 was used. Early stopping was done based on the validation loss. Pre-training modules one and two took at most 1.3 million iterations. Training the full model was at most 3.4 million iterations. When training the full model, the parameters for modules one and two were either randomly initialized, transferred from the pre-training step, or transferred and from the pre-training step and frozen (FIG. 23).

### De Novo Design of Zinc Finger Helix Pairs

When predicting zinc finger residues, the model makes use of context provided by known residues. Helix sequences are generated incrementally where the network is run once for each missing residue. At each iteration, a single residue is added to increase the sequence context. For a pair of helices, there are about 4.1×10¹⁵ possible sequences and about 4.8×10⁸ orders in which each sequence can be generated. Enumerating all possibilities to find the sequence with the highest likelihood is thus computationally intractable.

To generate sequences, we adapted the A* search algorithm, as done previously^{5,6}. This approach involves iteratively filling in masked residues while maintaining a priority queue of partially masked sequences. At every iteration, the top partially masked sequence is taken from the priority queue and passed through the network. All possible labels for every masked residue are evaluated. Any label with a probability above 0.05 is accepted and the label is added to a copy of the input sequence before it is pushed onto the priority queue. This is repeated until a set amount of sequences are completely generated. The following equation is used to assign a priority to each partially masked sequence:

$${p}_{j} = {\sum }_{i = 1}^{j} log ( {p}_{i} ) + {\sum }_{j}^{12} log ( {p}^{*} )$$

This heuristic approximates the maximum expected probability of a sequence that would be attained by predicting the remaining residues. pᵢ denotes the probability assigned to the prediction made at iteration i and j denotes the number of predicted residues. p* denotes the expected maximum probability that would be assigned by the network to later predictions. This parameter can be tuned to move the search closer to a greedy search or a breadth first search. This parameter was set to 0.1 whenever A* was performed in this work.

We also implemented an alternative biased sampling approach using temperature adjusted distributions, as done previously⁷. This approach generally resulted in higher likelihood sequences (FIG. 24). At every iteration, the probability of predicting an amino acid i at position j is the following:

$${p ( {x}_{i , j} \vert n , {x}_{( k , m ) ∈ S} )}^{( T )} = \frac{{p ( {x}_{i , j} \vert n , {x}_{( k , m ) ∈ S} )}^{\frac{1}{T}}}{{Σ}_{a = 1}^{20} {Σ}_{b = 1}^{12} {p ( {x}_{a , b} \vert n , {x}_{( k , m ) ∈ S} )}^{\frac{1}{T}}}$$

n denotes the input nucleotide sequence and S denotes the set of pairs of amino acids and positions that have already been predicted. T is an adjustable parameter that controls the bias of the distribution. This parameter was set to 0.6 when this method was used. 10⁵ ZF pairs were sampled and the maximum likelihood pair when performing de novo design.

### Comparison to ZFPred

To generate distributions over helix sequences using ZFPred³, 10⁶ helix sequences were randomly sampled. The binding specificities of these helices were predicted using ZFPred. Sequence distributions for a particular nucleotide sequence were then generated by normalizing the predicted scores of the sampled helices for that nucleotide sequence. Predictions for 3-mers were concatenated to generate predictions for 6-mer sequences.

### Live Cell Imagine of ZF-GFP Fusion

We designed zinc fingers to bind the sequence 5′-CGCCCAGCTGGGGGCGGGGGA-3′, a sequence that is repeated 111 times at the Brf1 locus on chromosome 14 (hg38 chr14:105229626-105240946). The coding sequence for the designed zinc finger array was ordered from IDT (gBlock) A SV40 NLS was added to the C-termini by PCR. Next, we added GFP as an N-terminal fusion to the zinc fingers using the NT-GFP Fusion TOPO TA Expression Kit (Invitrogen). Successful cloning into the expression vector was confirmed by Sanger sequencing.

The GFP-ZF fusion expression vector was transfected into 293T cells and grown on 0.01% Poly-L-Lysine coated 35 mm MatTek dishes using X-treme-GENE 9 DNA transfection reagent (Sigma Aldrich). Transfected cells were Hoechst stained the next day and then imaged. A titration experiment was conducted to explore optimal plasmid concentration. Clear foci were visible at a range of concentrations, but 333 ng of plasmid yielded the optimal balance of transfection efficiency and signal to noise ratio.

### Cell Culture and RT-qPCR Analysis of Repressors and Activators

HEK293T cells were transfected with ZF-repressors, ZF-activators, or SpCas9-repressors targeting various endogenous loci and target transcript levels were measured by RT-qPCR as follows. 2 μg of the parental (pKJ-Kan) plasmid DNA or 2 μg of pMMBC_SpCas9 containing a non-targeting guide were used as negative controls for ZF and SpCas9 transfections, respectively. Cells were cultured in DMEM supplemented with 10% FBS, 2 mM GlutaMAX™ (Life Technologies), 1% penicillin/streptomycin, 1% MEM non-essential amino acids (Life Technologies) and 2 mM sodium pyruvate. 18-24 hours prior to transfection, cells were passaged and 7.5e5 cells were added to 2.5 mL media in a 6-well dish. Cells were transfected with 2 μg of plasmid DNA using a 4:1 ratio of DNA: TransIT®-LT1 transfection reagent (Mirus) according to manufacturer's instructions. Media was changed 2 days post-transfection, and cells were harvested for RT-qPCR 3 days post-transfection. Cells were washed once with sterile PBS, 350 μL Buffer RLT Plus (Qiagen) containing 1% β-mercaptoethanol was added, and samples were either stored at −80 C or processed immediately using a RNeasy Plus Mini Kit (Qiagen) according to manufacturer's instructions. Pure RNA was quantified using a NanoDrop™ 2000c (Thermo Scientific™) and stored at −80 C.

1 μg of pure RNA was reverse transcribed using the SuperScript™ IV First-Strand Synthesis System (Invitrogen™) according to manufacturer's instructions except half the recommended reverse transcriptase was used. Random hexamers were used as primers, and cDNA was stored at −20 C or processed immediately. qPCR reactions were set up in technical duplicate or triplicate using the equivalent of 25 ng or 50 ng reverse-transcribed RNA per reaction and the KAPA SYBR FAST qPCR Master Mix (2X) (Roche).

RT-qPCR was performed on a LightCycler® 480 Instrument II (Roche) using the cycling program recommended for KAPA SYBR FAST reagent on the LightCycler® 480 (annealing temperature was 60 C). Ct values were calculated using the on-board “Absolute Quantification/2^{nd} Derivative Max” analysis option. Input was first normalized using the housekeeping gene RPS18, and fold-change in expression for a given gene of interest was calculated relative to the appropriate negative control. A table of RT-qPCR primers used in this study can be found in the supplementary data.

### RNA-seq Analysis

RNA-Seq library preps were constructed using the Illumina TruSeq® Stranded mRNA Library Prep kit (Cat #20020595) using 500-1000 ng of total RNA as input, amplified by 10-12 cycles of PCR, and sequenced paired-end 50 cycles on Illumina sequencers with 2% PhiX spike-in. 25-30 million reads were obtained for each sample. Paired-end reads were aligned to hg38 using STAR aligner⁸. Read counts were computed using FeatureCounts and differential expression analysis was subsequently performed using DESeq2⁹.

### Statistical Analysis

Two-sided Wilcoxon rank-sum tests were performed using the SciPy python library. Boxplot centerlines show medians, box limits show upper and lower quartiles, whiskers are 1.5 the interquartile range and points show outliers.

This reference listing is not an indication that any particular reference is material to patentability:
1. N. Matharu et al., CRISPR-mediated activation of a promoter or enhancer rescues obesity caused by haploinsufficiency. Science 363, (2019).
2. A. A. Dominguez, W. A. Lim, L. S. Qi, Beyond editing: repurposing CRISPR-Cas9 for precision genome regulation and interrogation. Nat Rev Mol Cell Biol 17, 5-15 (2016).
3. B. Chen, R. B. Altman, Opportunities for developing therapies for rare genetic diseases: focus on gain-of-function and allostery. Orphanet J Rare Dis 12, 61 (2017).
4. L. A. Gilbert et al., Genome-Scale CRISPR-Mediated Control of Gene Repression and Activation. Cell 159, 647-661 (2014).
5. P. Perez-Pinera et al., RNA-guided gene activation by CRISPR-Cas9-based transcription factors. Nat Methods 10, 973-976 (2013).
6. P. I. Thakore, C. A. Gersbach, Design, Assembly, and Characterization of TALE-Based Transcriptional Activators and Repressors. Methods Mol Biol 1338, 71-88 (2016).
7. P. I. Thakore et al., Highly specific epigenome editing by CRISPR-Cas9 repressors for silencing of distal regulatory elements. Nat Methods 12, 1143-1149 (2015).
8. A. Amabile et al., Inheritable Silencing of Endogenous Genes by Hit-and-Run Targeted Epigenetic Editing. Cell 167, 219-232 e214 (2016).
9. J. K. Nunez et al., Genome-wide programmable transcriptional memory by CRISPR-based epigenome editing. Cell 184, 2503-2519 e2517 (2021).
10. M. Jinek et al., A programmable dual-RNA-guided DNA endonuclease in adaptive bacterial immunity. Science 337, 816-821 (2012).
11. C. T. Charlesworth et al., Identification of preexisting adaptive immunity to Cas9 proteins in humans. Nat Med 25, 249-254 (2019).
12. D. L. Wagner et al., High prevalence of Streptococcus pyogenes Cas9-reactive T cells within the adult human population. Nat Med 25, 242-248 (2019).
13. C. Anders, O. Niewoehner, A. Duerst, M. Jinek, Structural basis of PAM-dependent target DNA recognition by the Cas9 endonuclease. Nature 513, 569-573 (2014).
14. H. Nishimasu et al., Crystal structure of Cas9 in complex with guide RNA and target DNA. Cell 156, 935-949 (2014).
15. I. Sadowski, J. Ma, S. Triezenberg, M. Ptashne, GAL4-VP16 is an unusually potent transcriptional activator. Nature 335, 563-564 (1988).
16. A. Chavez et al., Highly efficient Cas9-mediated transcriptional programming. Nat Methods 12, 326-328 (2015).
17. C. C. Wilkens MS, Pearl J, Schanzer E, Liao H, Van Biber B, Quietsch K, Bloom J, Federation A, Acosta R, Vong S, Otterman E, Dunn D, Wang H, Zraszhevskiy P, Nandakumar V, Bates D, Sandstrom R, Urnov FD, Funnell A, Green S, and Stamatoyannopoulos JA, Quantitative dialing of gene expression via precision targeting of KRAB repressors. BioRxiv, (2021).
18. S. A. Wolfe, L. Nekludova, C. O. Pabo, DNA recognition by Cys2His2 zinc finger proteins. Annu Rev Biophys Biomol Struct 29, 183-212 (2000).
19. A. Klug, The discovery of zinc fingers and their applications in gene regulation and genome manipulation. Annu Rev Biochem 79, 213-231 (2010).
20. S. A. Lambert et al., The Human Transcription Factors. Cell 175, 598-599 (2018).
21. M. Imbeault, P. Y. Helleboid, D. Trono, KRAB zinc-finger proteins contribute to the evolution of gene regulatory networks. Nature 543, 550-554 (2017).
22. S. V. Razin, V. V. Borunova, O. G. Maksimenko, O. L. Kantidze, Cys2His2 zinc finger protein family: classification, functions, and major members. Biochemistry (Mosc) 77, 217-226 (2012).
23. S. Sydor et al., Kruppel-like factor 6 is a transcriptional activator of autophagy in acute liver injury. Sci Rep 7, 8119 (2017).
24. H. A. Greisman, C. O. Pabo, A general strategy for selecting high-affinity zinc finger proteins for diverse DNA target sites. Science 275, 657-661 (1997).
25. M. Isalan, A. Klug, Y. Choo, A rapid, generally applicable method to engineer zinc fingers illustrated by targeting the HIV-1 promoter. Nat Biotechnol 19, 656-660 (2001).
26. D. J. Segal, B. Dreier, R. R. Beerli, C. F. Barbas, 3rd, Toward controlling gene expression at will: selection and design of zinc finger domains recognizing each of the 5′-GNN-3′ DNA target sequences. Proc Natl Acad Sci USA 96, 2758-2763 (1999).
27. M. L. Maeder et al., Rapid “open-source” engineering of customized zinc-finger nucleases for highly efficient gene modification. Mol Cell 31, 294-301 (2008).
28. A. Gupta et al., An optimized two-finger archive for ZFN-mediated gene targeting. Nat Methods 9, 588-590 (2012).
29. Y. Choo, A. Klug, Toward a code for the interactions of zinc fingers with DNA: selection of randomized fingers displayed on phage. Proc Natl Acad Sci USA 91, 11163-11167 (1994).
30. B. Dreier, R. R. Beerli, D. J. Segal, J. D. Flippin, C. F. Barbas, 3rd, Development of zinc finger domains for recognition of the 5′-ANN-3′ family of DNA sequences and their use in the construction of artificial transcription factors. J Biol Chem 276, 29466-29478 (2001).
31. B. Dreier et al., Development of zinc finger domains for recognition of the 5′-CNN-3′ family DNA sequences and their use in the construction of artificial transcription factors. J Biol Chem 280, 35588-35597 (2005).
32. E. J. Rebar, C. O. Pabo, Zinc finger phage: affinity selection of fingers with new DNA-binding specificities. Science 263, 671-673 (1994).
33. C. Zhu et al., Using defined finger-finger interfaces as units of assembly for constructing zinc-finger nucleases. Nucleic Acids Res 41, 2455-2465 (2013).
34. T. Kim et al., MUSI: an integrated system for identifying multiple specificity from very large peptide or nucleic acid data sets. Nucleic Acids Res 40, e47 (2012).
35. A. L. Mueller et al., The geometric influence on the Cys2His2 zinc finger domain and functional plasticity. Nucleic Acids Res 48, 6382-6402 (2020).
36. A. R. Leach, A. P. Lemon, Exploring the conformational space of protein side chains using dead-end elimination and the A* algorithm. Proteins 33, 227-239 (1998).
37. G. V. Ingraham J, Barzilay R, and Jaakkola T., in Advnaces of Neural Information Processing Systems 32. (2019).
38. E. M. Handel et al., Versatile and efficient genome editing in human cells by combining zinc-finger nucleases with adeno-associated viral vectors. Hum Gene Ther 23, 321-329 (2012).
39. D. Reyon et al., FLASH assembly of TALENs for high-throughput genome editing. Nat Biotechnol 30, 460-465 (2012).
40. B. P. Kleinstiver et al., Engineered CRISPR-Cas9 nucleases with altered PAM specificities. Nature 523, 481-485 (2015).
41. D. E. Paschon et al., Diversifying the structure of zinc finger nucleases for high-precision genome editing. Nat Commun 10, 1133 (2019).
42. M. S. Bhakta et al., Highly active zinc-finger nucleases by extended modular assembly. Genome Res 23, 530-538 (2013).
43. N. Alerasool, D. Segal, H. Lee, M. Taipale, An efficient KRAB domain for CRISPRi applications in human cells. Nat Methods 17, 1093-1096 (2020).
44. A. S. Khalil et al., A synthetic biology framework for programming eukaryotic transcription functions. Cell 150, 647-658 (2012).
45. J. C. Miller et al., Enhancing gene editing specificity by attenuating DNA cleavage kinetics. Nat Biotechnol 37, 945-952 (2019).
46. A. V. Persikov, E. F. Rowland, B. L. Oakes, M. Singh, M. B. Noyes, Deep sequencing of large library selections allows computational discovery of diverse sets of zinc fingers that bind common targets. Nucleic Acids Res 42, 1497-1508 (2014).
47. A. L. Mueller et al., The geometric influence on the Cys2His2 zinc finger domain and functional plasticity. Nucleic Acids Research 48, 6382-6402 (2020).
48. M. Garton et al., A structural approach reveals how neighbouring C2H2 zinc fingers influence DNA binding specificity. Nucleic Acids Research 43, 9147-9157 (2015).
49. M. Elrod-Erickson, M. A. Rould, L. Nekludova, C. O. Pabo, Zif268 protein& #x2013; DNA complex refined at 1.6& #xe5;: a model system for understanding zinc finger& #x2013;DNA interactions. Structure 4, 1171-1180 (1996).
50. D. A. Case et al., The Amber biomolecular simulation programs. Journal of computational chemistry 26, 1668-1688 (2005).

1. A modified protein comprising an introduced zinc finger DNA binding domain, the modified protein having a changed DNA binding location relative to a DNA binding location of the protein in its unmodified form, wherein the modified protein comprises, in addition to the introduced zinc finger DNA binding domain, a gene expression activator domain or a gene expression repressor domain.

2. The modified protein of claim 1, wherein the changed DNA binding location is on a chromosome, organelle DNA, or a plasmid.

3. The modified protein of claim 1, comprising the gene expression activator domain, wherein DNA binding of the modified protein promotes expression of a gene that is operably linked to the changed DNA binding domain location.

4. The modified protein of claim 3, wherein the gene expression activator domain comprises a Krueppel-like factor 6 (KLF) protein or functional segment thereof.

5. The modified protein of claim 1, comprising the gene expression repressor domain, and wherein DNA binding of the modified protein inhibits expression of a gene that is operably linked to the changed DNA binding domain location, to thereby inhibit expression of the gene.

6. The modified protein of claim 5, the modified protein comprising the gene expression repressor domain, the gene expression domain comprising a KRAB domain, wherein the KRAB domain is optionally comprised by a Zim3 protein or functional segment thereof.

7. The modified protein of claim 1, wherein the introduced zinc finger binding domain comprises a substitution of an endogenous zinc finger domain of the protein in its unmodified form.

8. The modified protein of claim 7, wherein the introduced zinc finger domain is one of a plurality of zinc finger domains that are introduced into the modified protein to thereby provide a modified protein comprising a plurality of introduced zinc finger domains, and wherein the plurality of introduced zinc finger domains optionally comprise the same changed DNA binding domain.

9. A cDNA or mRNA encoding a modified protein of claim 1.

10. The cDNA or mRNA of claim 9, wherein the introduced zinc finger domain is one of a plurality of zinc finger domains that are introduced into the modified protein to thereby provide a modified protein comprising a plurality of introduced zinc finger domains, and wherein the plurality of introduced zinc finger domains optionally comprise the same changed DNA binding domain.

11. An expression vector encoding a modified protein of claim 1.

12. The expression vector of claim 11, wherein the introduced zinc finger domain is one of a plurality of zinc finger domains that are introduced into the modified protein to thereby provide a modified protein comprising a plurality of introduced zinc finger domains, and wherein the plurality of introduced zinc finger domains optionally comprise the same changed DNA binding domain.

13. A pharmaceutical composition comprising: i) one or more modified proteins of claim 1; ii) one or more mRNAs encoding one or more of said modified proteins; or iii) one or more expression vectors encoding one or more of said modified proteins, and wherein optionally the introduced zinc finger domain is one of a plurality of zinc finger domains that are introduced into the modified protein to thereby provide a modified protein comprising a plurality of introduced zinc finger domains, and wherein the plurality of introduced zinc finger domains optionally comprise the same changed DNA binding domain.

14. A method comprising administering to an individual in need thereof a pharmaceutical composition of claim 13.

15. The method of claim 14, wherein the individual is in need of activation or repression of one or more genes, wherein the modified protein promotes activation or repression of said one or more genes, respectively.

16. The method of claim 15, wherein an immune response to the modified protein is not stimulated in the individual.

17. A method of making a modified protein of claim 1, the method comprising modifying a protein to comprise an introduced zinc finger DNA binding domain.

18. The method of claim 17, wherein the protein that is modified comprises a gene expression activator or gene expression repressor domain.

19. The method of claim 18, wherein the modified protein is produced by cells comprising an expression vector encoding the modified protein, the method further comprising separating the modified protein from said cells.

20. Cells comprising a modified protein of claim 1.


SEQUENCE LISTING (95 sequences)

SEQ ID NO: 1 - protein, 12 aa, artificial sequence
  zinc finger fusion protein fragment
NSTALQARNDSR

SEQ ID NO: 2 - protein, 9 aa, artificial sequence
  zinc finger fusion protein fragment
NNNACAAAG

SEQ ID NO: 3 - protein, 9 aa, artificial sequence
  zinc finger fusion protein fragmen
NNNACAAAG

SEQ ID NO: 4 - protein, 6 aa, artificial sequence
  zinc finger fusion protein fragment
RSDNRA

SEQ ID NO: 5 - protein, 6 aa, artificial sequence
  zinc finger fusion protein fragment
QLATSN

SEQ ID NO: 6 - protein, 6 aa, artificial sequence
  zinc finger fusion protein fragment
DQSNTR

SEQ ID NO: 7 - protein, 6 aa, artificial sequence
  zinc finger fusion protein fragment
FQSGIQ

SEQ ID NO: 8 - protein, 6 aa, artificial sequence
  zinc finger fusion protein fragment
HKRNTD

SEQ ID NO: 9 - protein, 6 aa, artificial sequence
  zinc finger fusion protein fragment
DQSALG

SEQ ID NO: 10 - protein, 6 aa, artificial sequence
  zinc finger fusion protein fragment
TKQNTH

SEQ ID NO: 11 - protein, 6 aa, artificial sequence
  zinc finger fusion protein fragment
QLATSY

SEQ ID NO: 12 - protein, 6 aa, artificial sequence
  zinc finger fusion protein fragment
RNGNTR

SEQ ID NO: 13 - protein, 6 aa, artificial sequence
  zinc finger fusion protein fragment
YQPNIN

SEQ ID NO: 14 - DNA, 21 bp, artificial sequence
  zinc finger target sequence
agtcgcccagctgggggcggg

SEQ ID NO: 15 - protein, 40 aa, artificial sequence
  zinc finger fusion protein fragment
ACDEFGHIKLMNPQRSTVWYACDEFGHIKLMNPQRSTVWY

SEQ ID NO: 16 - protein, 8 aa, artificial sequence
  zinc finger fusion protein fragment
AEKNQRTV

SEQ ID NO: 17 - protein, 7 aa, artificial sequence
  zinc finger fusion protein fragment
ADHNSTV

SEQ ID NO: 18 - protein, 8 aa, artificial sequence
  zinc finger fusion protein fragment
ADHKNQRS

SEQ ID NO: 19 - protein, 4 aa, artificial sequence
  zinc finger fusion protein fragment
NKRS

SEQ ID NO: 20 - protein, 7 aa, artificial sequence
  zinc finger fusion protein fragment
ADHNQRT

SEQ ID NO: 21 - protein, 8 aa, artificial sequence
  zinc finger fusion protein fragment
AEKNQRTV

SEQ ID NO: 22 - protein, 6 aa, artificial sequence
  zinc finger fusion protein fragment
DHNSTV

SEQ ID NO: 23 - protein, 7 aa, artificial sequence
  zinc finger fusion protein fragment
RSDNLRA

SEQ ID NO: 24 - protein, 7 aa, artificial sequence
  zinc finger fusion protein fragment
RSDNLRA

SEQ ID NO: 25 - protein, 7 aa, artificial sequence
  zinc finger fusion protein fragment
QLATLSN

SEQ ID NO: 26 - protein, 7 aa, artificial sequence
  zinc finger fusion protein fragment
DQSNLTR

SEQ ID NO: 27 - protein, 7 aa, artificial sequence
  zinc finger fusion protein fragment
DQSNLTR

SEQ ID NO: 28 - protein, 7 aa, artificial sequence
  zinc finger fusion protein fragment
FQSGLIQ

SEQ ID NO: 29 - protein, 7 aa, artificial sequence
  zinc finger fusion protein fragment
HKRNLTD

SEQ ID NO: 30 - protein, 7 aa, artificial sequence
  zinc finger fusion protein fragment
DQSALLG

SEQ ID NO: 31 - protein, 7 aa, artificial sequence
  zinc finger fusion protein fragment
TKQNLTH

SEQ ID NO: 32 - protein, 7 aa, artificial sequence
  zinc finger fusion protein fragment
QLATLSY

SEQ ID NO: 33 - protein, 7 aa, artificial sequence
  zinc finger fusion protein fragment
YQPNLIN

SEQ ID NO: 34 - protein, 6 aa, artificial sequence
  zinc finger fusion protein fragment
ARNDSR

SEQ ID NO: 35 - protein, 6 aa, artificial sequence
  zinc finger fusion protein fragment
ARNDSR

SEQ ID NO: 36 - protein, 6 aa, artificial sequence
  zinc finger fusion protein fragment
NSTALQ

SEQ ID NO: 37 - protein, 6 aa, artificial sequence
  zinc finger fusion protein fragment
RTNSQD

SEQ ID NO: 38 - protein, 7 aa, artificial sequence
  zinc finger fusion protein fragment
RNTNSQD

SEQ ID NO: 39 - protein, 6 aa, artificial sequence
  zinc finger fusion protein fragment
QIGSQF

SEQ ID NO: 40 - protein, 6 aa, artificial sequence
  zinc finger fusion protein fragment
DTNRKH

SEQ ID NO: 41 - protein, 6 aa, artificial sequence
  zinc finger fusion protein fragment
GLASQD

SEQ ID NO: 42 - protein, 6 aa, artificial sequence
  zinc finger fusion protein fragment
HTNQKT

SEQ ID NO: 43 - protein, 6 aa, artificial sequence
  zinc finger fusion protein fragment
YSTALQ

SEQ ID NO: 44 - protein, 6 aa, artificial sequence
  zinc finger fusion protein fragment
RTNGNR

SEQ ID NO: 45 - protein, 6 aa, artificial sequence
  zinc finger fusion protein fragment
NINPQY

SEQ ID NO: 46 - DNA, 22 bp, artificial sequence
  zinc finger target sequence
gtctctatcactgatagggaga

SEQ ID NO: 47 - DNA, 21 bp, artificial sequence
  zinc finger target sequence
gtctctatcactgatagggag

SEQ ID NO: 48 - DNA, 21 bp, artificial sequence
  zinc finger target sequence
tctctatcactgatagggaga

SEQ ID NO: 49 - DNA, 22 bp, artificial sequence
  zinc finger target sequence
tctccctatcagtgatagagac

SEQ ID NO: 50 - DNA, 21 bp, artificial sequence
  zinc finger target sequence
tctccctatcagtgatagaga

SEQ ID NO: 51 - DNA, 21 bp, artificial sequence
  zinc finger target sequence
ctccctatcagtgatagagac

SEQ ID NO: 52 - protein, 42 aa, artificial sequence
  zinc finger fusion protein fragment
QKVHLQSRKWTLSVRKGTLQDQYSSLYKRKGDLNKDPSSLRR

SEQ ID NO: 53 - protein, 42 aa, artificial sequence
  zinc finger fusion protein fragment
RKYNLLRRRYSLSAQKAHLLSDPSNLRRQKRLLQNWKVDLRK

SEQ ID NO: 54 - protein, 42 aa, artificial sequence
  zinc finger fusion protein fragment
RKFNLLRQSNTLRTLKHHLLNTSSGLCHEKRTLLNWKVDLRK

SEQ ID NO: 55 - protein, 42 aa, artificial sequence
  zinc finger fusion protein fragment
QKTHLLTRRDYLTKRKFTLLRQSNDLRKLKQTLQDRRDRLRR

SEQ ID NO: 56 - protein, 341 aa, artificial sequence
  synthetic zinc finger
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLAREVPSINKETLTTQKGVECDGSKKILPLGIDDVSSLQHYVQNNSHDDNGYRKLVGNNPSKFVGQQFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHSR

SEQ ID NO: 57 - protein, 373 aa, artificial sequence
  synthetic zinc finger
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHL

SEQ ID NO: 58 - protein, 471 aa, artificial sequence
  synthetic zinc finger
MDAKSLTAWSRTLVTFKDVFVDFTREEWKLLDTAQQIVYRNVMLENYKNLVSLGYQLTKPDVILRLEKGEEPWLVEREIHQETHPDSETAFEIKSSVSSRSIFKDKQSCDIKMEGMARNDLWYLSLEEVWKCRDQLDKYQENPERHLRQVAFTQKKVLTQERVSESGKYGGNCLLPAQLVLREYFHKRDSHTKSLKHDLVLNGHQDSCASNSNECGQTFCQNIHLIQFARTHTGDKSYKCPDNDNSLTHGSSLGISKGIHREKPFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHTGEQFLTCNQCGTALVNTSNLIGYQTNHIRENAY

SEQ ID NO: 59 - protein, 441 aa, artificial sequence
  synthetic zinc finger
MAAAVLTDRAQVSVTFDDVAVTFTKEEWGQLDLAQRTLYQEVMLENCGLLVSLGCPVPKAELICHLEHGQEPWTRKEDLSQDTCPGDKGKPKTTEPTTCEPALSEGISLQGQVTQGNSVDSQLGQAEDQDGLSEMQEGHFRPGIDPQEKSPGKMSPECDGLGTADGVCSRIGQEQVSPGDRVRSHNSCESGKDPMIQEEENNFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHTGKNPISVTDVGRPFTSGQTSVTLRELLLGKDFLNVTTEANILPEETSSSASDQPYQRETPQVSSL

SEQ ID NO: 60 - protein, 479 aa, artificial sequence
  synthetic zinc finger
MAFEDVAVYFSQEEWGLLDTAQRALYRRVMLDNFALVASLGLSTSRPRVVIQLERGEEPWVPSGTDTTLSRTTYRRRNPGSWSLTEDRDVSGEWPRAFPDTPPGMTTSVFPVAGACHSVKSLQRQRGASPSRERKPTGVSVIYWERLLLGSGSGQASVSLRLTSPLRPPEGVRLREKTLTEHALLGRQPRTPERQKPCAQEVPGRTFGSAQDLEAAGGRGHHRMGAVWQEPHRLLGGQEPSTWDELGEALHAGEKSFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHTGEKTVRRSRASLHPQARSVAGASSEGAPAKETEPTPASGPAAVSQPAEV

SEQ ID NO: 61 - protein, 392 aa, artificial sequence
  synthetic zinc finger
MDVLASYSIFQELQLVHDTGYFSALPSLEETWQQTCLELERYLQTEPRRISETFGEDLDCFLHASPPPCIEESFRRLDPLLLPVEAAICEKSSAVDILLSRDKLLSETCLSLQPASSSLDSYTAVNQAQLNAVTSLTPPSSPELSRHLVKTSQTLSAVDGTVTLKLVAKKAALSSVKVGGVATAAAAVTAAGAVKSGQSDSDQGGLGAEACPENKKRVFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHI

SEQ ID NO: 62 - protein, 379 aa, artificial sequence
  synthetic zinc finger
MDLKLKDCEFWYSLHGQVPGLLDWDMRNELFLPCTTDQCSLAEQILAKYRVGVMKPPEMPQKRRPSPDGDGPPCEPNLWMWVDPNILCPLGSQEAPKPSGKEDLTNISPFPQPPQKDEGSNCSEDKVVESLPSSSSEQSPLQKQGIHSPSDFELTEEEAEEPDDNSLQSPEMKCYQSQKLWQINNQEKSFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHIQECMSQPELLTSLFDL

SEQ ID NO: 63 - protein, 729 aa, artificial sequence
  synthetic zinc finger
MDLPALLPAPTARGGQHGGGPGPLRRAPAPLGASPARRRLLLVRGPEDGGPGARPGEASGPSPPPAEDDSDGDSFLVLLEVPHGGAAAEAAGSQEAEPGSRVNLASRPEQGPSGPAAPPGPGVAPAGAVTISSQDLLVRLDRGVLALSAPPGPATAGAAAPRRAPQASGPSTPGFACDICGRKFARKFNLLRHTRIHTGEKPFACDICGRKFAQSNTLRTHTKIHTQRPQIPPKPFACDICGRKFALKHHLLNHTRIHTGEKPFACDICGRKFATSSGLCHHTKIHTQRPQIPPKPFACDICGRKFAEKRTLLNHTRIHTGEKPFACDICGRKFAWKVDLRKHTKIHSRRQDLLPQLEAPSSLTPSSELSSPGQSELTNMDLAALFSDTPANASGSAGGSDEALNSGILTIDVTSVSSSLGGNLPANNSSLGPMEPLVLVAHSDIPPSLDSPLVLGTAATVLQQGSFSVDDVQTVSAGALGCLVALPMKNLSDDPLALTSNSNLAAHITTPTSSSTPRENASVPELLAPIKVEPDSPSRPGAVGQQEGSHGLPQSTLPSPAEQHGAQDTELSAGTGNFYLESGGSARTDYRAIQLAKEKKQRGAGSNAGASQSTQRKIKEGKMSPPHFHASQNSWLCGSLVVPSGGRPGPAPAAGVQCGAQGVQVQLVQDDPSGEGVLPSARGPATFLPFLTVDLPVYVLQEVLPSSGGPAGPEATQFPGSTINLQDLQ

SEQ ID NO: 64 - protein, 56 aa, artificial sequence
  zinc finger fusion protein fragment
RKWNLLMRSTNLRDYPYLLRNERSKLRRRVDTLLDHLSNLRKDPSALIRRLDVLRA

SEQ ID NO: 65 - protein, 402 aa, artificial sequence
  synthetic zinc finger
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLAREVPSINKETLTTQKGVECDGSKKILPLGIDDVSSLQHYVQNNSHDDNGYRKLVGNNPSKFVGQQFACDICGRKFARKWNLLMHTRIHTGEKPFACDICGRKFARSTNLRDHTKIHTQRPQIPPKPFACDICGRKFAYPYLLRNHTRIHTGEKPFACDICGRKFAERSKLRRHTKIHTQRPQIPPKPFACDICGRKFARVDTLLDHTRIHTGEKPFACDICGRKFAHLSNLRKHTKIHTQRPQIPPKPFACDICGRKFADPSALIRHTRIHTGEKPFACDICGRKFARLDVLRAHTKIHSR

SEQ ID NO: 66 - DNA, 28 bp, artificial sequence
  zinc finger target sequence
ctggtcgtatccggggcagcggagcagg

SEQ ID NO: 67 - DNA, 28 bp, artificial sequence
  zinc finger target sequence
gccaatgggcggtgcgcgggggccgggc

SEQ ID NO: 68 - DNA, 28 bp, artificial sequence
  zinc finger target sequence
ggccgcggcggggcggggcagcggggcg

SEQ ID NO: 69 - DNA, 28 bp, artificial sequence
  zinc finger target sequence
ggggcggccgccaatcgccgtggtgttg

SEQ ID NO: 70 - DNA, 28 bp, artificial sequence
  zinc finger target sequence
ttgaaactgaaaatactacattatgcta

SEQ ID NO: 71 - protein, 56 aa, artificial sequence
  zinc finger fusion protein fragment
RKYHLSRDRSTLRRRKDHLRNFPYLLRRLKHHLLRERSKLRRLKQTLQVDRSTLRR

SEQ ID NO: 72 - protein, 283 aa, artificial sequence
  synthetic zinc finger
MDVLPMCSIFQELQIVHETGYFSALPSLEEYWQQTCLELERYLQSEPCYVSASEIKFDSQEDLWTKIILAREKKEESELKISSSPPEDTLISPSFCYNLETNSLNSDVSSESSDSSEELSPTAKFTSDPIGEVLVSSGKLSSSVTSTPPSSPELSREPSQLWGCVPGELPSPGKVRSGTSGKPGDKGNGDASPDGRRRVHRCHFNGCRKVYTKSSHLKAHQRTHTGEKPYRCSWEGCEWRFARSDELTRHFRKHTGAKPFKCSHCDRCFSRSDHLALHMKRHL

SEQ ID NO: 73 - protein, 25 aa, artificial sequence
  zinc finger fusion protein fragment
HRCHFNGCRKVYTKSSHLKAHQRTH

SEQ ID NO: 74 - protein, 23 aa, artificial sequence
  zinc finger fusion protein fragment
FKCSHCDRCFSRSDHLALHMKRH

SEQ ID NO: 75 - protein, 23 aa, artificial sequence
  zinc finger fusion protein fragment
  misc_feature (12)..(15): Xaa can be any naturally occurring amino acid
  misc_feature (17)..(18): Xaa can be any naturally occurring amino acid
FQCRICMRNFSXXXXLXXHIRTH

SEQ ID NO: 76 - protein, 23 aa, artificial sequence
  zinc finger fusion protein fragment
  misc_feature (12)..(15): Xaa can be any naturally occurring amino acid
  misc_feature (17)..(18): Xaa can be any naturally occurring amino acid
FACDICGRKFAXXXXLXXHTKIH

SEQ ID NO: 77 - protein, 22 aa, artificial sequence
  zinc finger fusion protein fragment
  misc_feature (2)..(2): Xaa can be any naturally occurring amino acid
  misc_feature (4)..(5): Xaa can be any naturally occurring amino acid
  misc_feature (7)..(8): Xaa can be any naturally occurring amino acid
  misc_feature (10)..(14): Xaa can be any naturally occurring amino acid
  misc_feature (16)..(17): Xaa can be any naturally occurring amino acid
  misc_feature (19)..(21): Xaa can be any naturally occurring amino acid
ZXCXXCXXZXXXXXZXXHXXXH

SEQ ID NO: 78 - protein, 22 aa, artificial sequence
  zinc finger fusion protein fragment
  misc_feature (2)..(2): Xaa can be any naturally occurring amino acid
  misc_feature (4)..(5): Xaa can be any naturally occurring amino acid
  misc_feature (7)..(8): Xaa can be any naturally occurring amino acid
  misc_feature (10)..(14): Xaa can be any naturally occurring amino acid
  misc_feature (16)..(17): Xaa can be any naturally occurring amino acid
  misc_feature (19)..(21): Xaa can be any naturally occurring amino acid
ZXCXXCXXZXXXXXZXXHXXXH

SEQ ID NO: 79 - protein, 373 aa, artificial sequence; identical to SEQ ID NO: 57
  synthetic zinc finger

SEQ ID NO: 80 - protein, 472 aa, artificial sequence
  synthetic zinc finger
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLAREVPSINKETLTTQKGVECDGSKKILPLGIDDVSSLQHYVQNNSHDDNGYRKLVGNNPSKFVGQQLKCNACRKLFSSKSRLQSHLRRHACQKPFECHSCGRAFGEKWKLDKHQKTHAEERPYKCENCGNAYKQKSNLFQHQKMHTKEKPYQCKTCGKAFSWKSSCINHEKIHNAKKSYQCNECEKSFRQNSTLIQHKKVHTGQKPFQCTDCGKAFIYKSDLVKHQRIHTGEKPYKCSICEKAFSQKSNVIDHEKIHTGKRAYECDLCGNTFIQKKNLIQHKKIHTGEKPYECNRCGKAFFQKSNLHSHQKTHSGERTYRCSECGKTFIRKLNLSLHKKTHTGQKPYGCSECGKAFADRSYLVRHQKRIHSR

SEQ ID NO: 81 - protein, 23 aa, artificial sequence
  zinc finger fusion protein fragment
LKCNACRKLFSSKSRLQSHLRRH

SEQ ID NO: 82 - protein, 24 aa, artificial sequence
  zinc finger fusion protein fragment
YGCSECGKAFADRSYLVRHQKRIH

SEQ ID NO: 83 - protein, 23 aa, artificial sequence
  zinc finger fusion protein fragment
  misc_feature (12)..(15): Xaa can be any naturally occurring amino acid
  misc_feature (17)..(18): Xaa can be any naturally occurring amino acid
FQCRICMRNFSXXXXLXXHIRTH

SEQ ID NO: 84 - protein, 23 aa, artificial sequence
  zinc finger fusion protein fragmen
  misc_feature (12)..(15): Xaa can be any naturally occurring amino acid
  misc_feature (17)..(18): Xaa can be any naturally occurring amino acid
FACDICGRKFAXXXXLXXHTKIH

SEQ ID NO: 85 - protein, 22 aa, artificial sequence
  zinc finger fusion protein fragment
  misc_feature (2)..(2): Xaa can be any naturally occurring amino acid
  misc_feature (4)..(5): Xaa can be any naturally occurring amino acid
  misc_feature (7)..(8): Xaa can be any naturally occurring amino acid
  misc_feature (10)..(14): Xaa can be any naturally occurring amino acid
  misc_feature (16)..(17): Xaa can be any naturally occurring amino acid
  misc_feature (19)..(21): Xaa can be any naturally occurring amino acid
ZXCXXCXXZXXXXXZXXHXXXH

SEQ ID NO: 86 - protein, 22 aa, artificial sequence
  zinc finger fusion protein fragment
  misc_feature (2)..(2): Xaa can be any naturally occurring amino acid
  misc_feature (4)..(5): Xaa can be any naturally occurring amino acid
  misc_feature (7)..(8): Xaa can be any naturally occurring amino acid
  misc_feature (10)..(14): Xaa can be any naturally occurring amino acid
  misc_feature (16)..(17): Xaa can be any naturally occurring amino acid
  misc_feature (19)..(21): Xaa can be any naturally occurring amino acid
ZXCXXCXXZXXXXXZXXHXXXH

SEQ ID NO: 87 - protein, 471 aa, artificial sequence
  synthetic zinc finger
MNNSQGRVTFEDVTVNFTQGEWQRLNPEQRNLYRDVMLENYSNLVSVGQGETTKPDVILRLEQGKEPWLEEEEVLGSGRAEKNGDIGGQIWKPKDVKESLAREVPSINKETLTTQKGVECDGSKKILPLGIDDVSSLQHYVQNNSHDDNGYRKLVGNNPSKFVGQQLKCNACRKLFSSKSRLQSHLRRHACQKPFECHSCGRAFGEKWKLDKHQKTHAEERPYKCENCGNAYKQKSNLFQHQKMHTKEKPYQCKTCGKAFSWKSSCINHEKIHNAKKSYQCNECEKSFRQNSTLIQHKKVHTGQKPFQCTDCGKAFIYKSDLVKHQRIHTGEKPYKCSICEKAFSQKSNVIDHEKIHTGKRAYECDLCGNTFIQKKNLIQHKKIHTGEKPYECNRCGKAFFQKSNLHSHQKTHSGERTYRCSECGKTFIRKLNLSLHKKTHTGQKPYGCSECGKAFADRSYLVRHQKRIHS

SEQ ID NO: 88 - protein, 23 aa, artificial sequence
  zinc finger fusion protein fragment
LKCNACRKLFSSKSRLQSHLRRH

SEQ ID NO: 89 - protein, 24 aa, artificial sequence
  zinc finger fusion protein fragment
YGCSECGKAFADRSYLVRHQKRIH

SEQ ID NO: 90 - protein, 23 aa, artificial sequence
  zinc finger fusion protein fragment
  misc_feature (12)..(15): Xaa can be any naturally occurring amino acid
  misc_feature (17)..(18): Xaa can be any naturally occurring amino acid
FQCRICMRNFSXXXXLXXHIRTH

SEQ ID NO: 91 - protein, 23 aa, artificial sequence
  zinc finger fusion protein fragment
  misc_feature (12)..(15): Xaa can be any naturally occurring amino acid
  misc_feature (17)..(18): Xaa can be any naturally occurring amino acid
FACDICGRKFAXXXXLXXHTKIH

SEQ ID NO: 92 - protein, 22 aa, artificial sequence
  zinc finger fusion protein fragment
  misc_feature (2)..(2): Xaa can be any naturally occurring amino acid
  misc_feature (4)..(5): Xaa can be any naturally occurring amino acid
  misc_feature (7)..(8): Xaa can be any naturally occurring amino acid
  misc_feature (10)..(14): Xaa can be any naturally occurring amino acid
  misc_feature (16)..(17): Xaa can be any naturally occurring amino acid
  misc_feature (19)..(21): Xaa can be any naturally occurring amino acid
ZXCXXCXXZXXXXXZXXHXXXH

SEQ ID NO: 93 - protein, 22 aa, artificial sequence
  zinc finger fusion protein fragment
  misc_feature (2)..(2): Xaa can be any naturally occurring amino acid
  misc_feature (4)..(5): Xaa can be any naturally occurring amino acid
  misc_feature (7)..(8): Xaa can be any naturally occurring amino acid
  misc_feature (10)..(14): Xaa can be any naturally occurring amino acid
  misc_feature (16)..(17): Xaa can be any naturally occurring amino acid
  misc_feature (19)..(21): Xaa can be any naturally occurring amino acid
ZXCXXCXXZXXXXXZXXHXXXH

SEQ ID NO: 94 - protein, 341 aa, artificial sequence; identical to SEQ ID NO: 56
  synthetic zinc finger

SEQ ID NO: 95 - protein, 7 aa, artificial sequence
  zinc finger fusion protein fragment
RNGNLTR